(19) United States
(12) Patent Application Publication
(10) Pub. No.: US 2022/0168366 A1
(43) Pub. Date: Jun. 2, 2022

(54) NUTRACEUTICAL COMPOSITIONS

(71) Applicants: GlaxoSmithKline Biologicals SA, Rixensart (BE); Washington University, St. Louis, MO (US)

(72) Inventors: Michael J. BARRATT, St. Louis, MO (US); Nicolas Frederic DELAHAYE, Rockville, MD (US); Jeffrey I. GORDON, St. Louis, MO (US)

(73) Assignees: GlaxoSmithKline Biologicals SA, Rixensart (BE); Washington University, St. Louis, MO (US)

(21) Appl. No.: 17/434,990

(22) PCT Filed: Mar. 2, 2020
(86) PCT No.: PCT/IB2020/051766

§ 371 Date: Aug. 30, 2021

Related U.S. Application Data
(60) Provisional application No. 62/812,965, filed on Mar. 1, 2019.

(51) Int. Cl. A61K 35/748; A61K 35/742; A61K 35/741; A61K 47/46; A61K 31/455; A61K 31/4415; A61K 31/375; A61K 31/593; A61K 31/355; A61K 31/519; A61K 39/39; A61K 9/50; A61P 37/04

(52) U.S. Cl. CPC A61K 35/748; A61K 35/742; A61K 35/741; A61K 47/46; A61K 31/455; A61K 31/4415; A61K 2039/55594; A61K 31/593; A61K 31/355; A61K 31/519; A61K 39/39; A61K 9/5015; A61P 37/04; A61K 31/375

(57) ABSTRACT

A nutraceutical composition comprising prebiotics, probiotics, and/or synbiotics, spirulina, cereals and micronutrients for improving a person's health, and methods for boosting the immune system and improving vaccine effectiveness in vulnerable populations with the nutraceutical composition, including undernourished children, lactating and pregnant mothers in LDCs, the elderly, and persons with cancer or at risk of developing cancer.

### CROSS-REFERENCE TO RELATED APPLICATIONS

This application claims priority to U.S. Patent Application Ser. No. 62/812,965 filed Mar. 1, 2019, the contents of which are incorporated by reference herein.

### FIELD OF THE INVENTION

Nutrient formulations that include prebiotics, probiotics, and/or synbiotics and their use as nutritive oral adjuvants for enhancing immune response to vaccines in a subject.

### NAMES OF PARTIES TO A JOINT RESEARCH AGREEMENT

The invention described herein is the subject of a Research Collaboration Agreement between GlaxoSmithKline Biologicals SA and the Washington University in St Louis entered into on Dec. 15, 2015, and amended Sep. 18, 2017, Jul. 1, 2018, Nov. 1, 2018 and Aug. 1, 2019.

### BACKGROUND TO THE INVENTION

More than 100 million under-five children in the world are undernourished (FAO 2013 Hunger Report). The first 1000 days of a child's life disproportionately impact survival and immune development. Studies have shown that improving nutrition favorably increases immune development (Karacabey K, Ozdemir N (2012). The Effect of Nutritional Elements on the Immune System. J Obes Wt Loss Ther 2:152. doi:10.4172/2165-7904.1000152). Children in developing countries with high rates of enteropathogen exposure and undernutrition often exhibit poor responses to oral vaccination. Strategies aimed at improving vaccine efficacy in these vulnerable populations have had limited success. Oral vaccination schedules begin early in postnatal life when the gut microbiota, which plays a key role in mucosal immune system development, is undergoing maturation to an adult-like configuration. Perturbed microbiota development has been causally linked to undernutrition, raising the possibility that this perturbation contributes to impaired oral vaccine performance. Work has been done to provide food supplements to address malnutrition, but the idea of using nutrition to repair the gut microbiota and improve responses to vaccines, particularly orally administered vaccines, is an emerging concept.

Spirulina (Arthrospira genus, formerly classified in the Spirulina genus) is a filamentous microscopic blue-green algae (cyanobacteria) that forms tangled masses in warm alkaline lakes in Africa and Central and South America and is cultivated worldwide. The two most commonly utilized species of Spirulina are Arthrospira platensis and Arthrospira maxima. Spirulina has been shown to be a hepatoprotective agent (Jeyaprakash, K. and P. Chinnaswamy, 2005. Effect of Spirulina and Liv-52 on cadmium induced toxicity in albino rats. Indian J. Exp. Biol., 43: 773-781.), neuroprotective (Sharma, M. K., A. Sharma, A. Kumar and M. Kumar, 2007. Evaluation of protective efficacy of Spirulina fusiformis against mercury induced nephrotoxicity in Swiss albino mice. Food Chem. Toxicol., 45: 879-887), to be effective against inflammation (Coskun, Z. K., M. Kerem, N. Gurbuz, S. Omeroglu and H. Pasaoglu et al., 2011. The study of biochemical and histopathological effects of Spirulina in rats with TNBS-induced colitis. Bratislayske Lekarske Listy, 112: 235-243), to have antitumor activity (increase NK cytotoxicity—Akao, 2009; Nielsen, 2010; Hirahashi, 2002), antimicrobial activity (inhibit the growth of some Gram-negative and Gram-positive bacteria—Bhowmik, 2009), and to have antiviral activity against HIV (increase T helper lymphocytes (CD4+ counts) in HIV-infected patients accompanied by a decrease in the viral load—Teas, 2012; Ngo-Matip, 2015; Azabji-Kenfack, 2011), and also to provide antioxidant effects (Takeda, T., A. Yokota and S. Shigeoka, 1995. Resistance of photosynthesis to hydrogen peroxide in algae. Plant Cell Physiol., 36: 1089-1095).

Arthrospira plantensis is also used as a food source or dietary supplement in humans because it provides a quality source of protein in good quantity, provides essential amino acids, and provides minerals, vitamins and polyunsaturated fatty acids. Spirulina is considered a GRAS micro-organism (Generally Recognized as Safe) with no toxicity, and is approved as a food additive by the FDA (Food and Drug Administration) without risks to health (Parisi et al., 2009; Ambrosi et al., 2008). Several researchers (Miranda et al., 1998; Herrero et al., 2005; Souza et al., 2006; Mendiola et al., 2007; Bierhals et al., 2009) have reported the nutritional and functional importance of the compounds present in Spirulina platensis (spirulina).

Probiotics have been shown to improve digestion and to act as an adjuvant in immune therapy (Licciardi et al. Discov Med 2011; Maidens et al. Br J Clin Pharmacol 2013; Valdez et al. Trends Immunol 2014). Numerous species of bacteria have been found to provide positive effects and act as probiotics, including Bacillus sp., Bifidobacterium sp., Enterococcus sp., Lactobacillus sp., Lactococcus sp., Pediococcus sp., Saccharomycessp. and Streptococcussp. Such probiotic species are available marketed as dietary supplements and are available to consumers in powder, tablet, capsule, caplet, gel, beadlet, some in controlled release formulations, and in liquid form. Probiotics are also present in certain foods such as yogurt, sauerkraut, buttermilk, kefir, miso and juice, either naturally, or added as a supplement.

Encapsulation is also a way to protect bioactive agents from digestion in the stomach whereby the bioactive agent is encapsulated or coated with an emulsion or microemulsion. Such encapsulated bioactive agents show significant levels of absorption in the gastrointestinal tract, leading to increased bioactivity and bioavailability (Takahashi et al., 2007), and have high biocompatibility and versatility.

Several studies have demonstrated differences among strains of probiotic bacteria with regard to their survival in acidic environments. Probiotics must survive in gastric acids to reach the small intestine and colonize the host for appropriate prevention and management of several gastrointestinal diseases. To improve the survival rates of probiotic microorganisms during gastric transit, microencapsulation is considered to be a promising process. A variety of polymers may be used for microencapsulation but there is a need for improved physical and mechanical stability of the polymers used in probiotic microencapsulation. Toward that end, milk proteins have begun to be used in probiotic microencapsulation formulas.

Microencapsulation is a process in which the probiotic cells are incorporated into an encapsulating matrix or membrane that can protect the cells from degradation by the damaging factors in the environment and release at controlled rates under particular conditions. One purpose of microencapsulation of probiotics is to protect them from the low pH, bile salts, and other constituent products that are encountered during gastrointestinal transit. A microcapsule comprises a semipermeable or nonpermeable, spherical, thin and strong membrane surrounding a solid and/or liquid core with very small diameter, varying between a few microns and 1 mm. Desirable encapsulation materials for a nutraceutical include those generally recognized as safe (GRAS), i.e. ingredients that can be used in food preparation.

### SUMMARY OF THE INVENTION

In an embodiment of the invention there is provided a nutraceutical composition comprising a probiotic species selected from Bacillus sp., Bifidobacterium sp., Enterococcus sp., Lactobacillus sp., Lactococcus sp., Propionibacterium sp., Pediococcus sp., Saccharomyces sp. and Streptococcus a Bacteroides, a Clostridioides, a Clostridium, an Erysipelotrichaceae, a Firmicutes, a Flavonifractor, a Fusobacterium, a Lactobacillus, a Parabacteroides, a Peptoclostridrium, a Robinsoniella, or a Subdoligranulum species; spirulina, a cereal and micronutrients.

One embodiment of the invention provides a nutraceutical composition comprising a probiotic species selected from a Bacteroides, a Clostridioides, a Clostridium, an Erysipelotrichaceae, a Firmicutes, a Flavonifractor, a Fusobacterium, a Lactobacillus, a Parabacteroides, a Peptoclostridrium, a Robinsoniella, or a Subdoligranulum species; spirulina, a cereal and micronutrients.

One embodiment of the invention provides a method of enhancing an immune response to a vaccine or antigen in a human, the method comprising administering an immune enhancing effective amount of a nutraceutical composition comprising: a probiotic species selected from Lactobacillus rhamnosus, Lactobacillus acidophilus; spirulina; cereal comprising flaxseed and amaranth; and micronutrients comprising i) vitamins B3, B6, C, D3, E and B9 and ii) minerals comprising magnesium, selenium and zinc; such that an enhanced immune response to a vaccine or antigen is observed in the human, as measured by an increase in mucosal IgA titer or as measured by an increase in systemic IgG titer.

One embodiment of the invention provides a method of using a probiotic to enhance the immune system in a human, the method comprising administering an immune enhancing effective amount of a probiotic species, wherein the probiotic species is a Bacteroides species, a Clostridioides species, Clostridium species, an Erysipelotrichaceae species, a Firmicutes species, a Flavonifractor species, a Fusobacterium species, a Lactobacillus species, a Parabacteroides species, a Peptoclostridrium species, a Robinsoniella species, a Subdoligranulum species or a combination thereof, such that the enhanced immune system is observable by an increased response to a vaccine or antigen in the human, as measured by an increase in mucosal IgA titer or as measured by an increase in systemic IgG titer.

One embodiment of the invention provides use of a nutraceutical composition as described herein for enhancing the immune system in a human by increasing an immune response to an antigen or a vaccine in said human, as measured by an increase in mucosal IgA titer or as measured by an increase in systemic IgG titer.

### DESCRIPTION OF DRAWINGS/FIGURES

FIG. 1A and FIG. 1B show the designs of Study 1 and Study 2, respectively, of germ free mice receiving microbiota from undernourished human donors fed Probiotic (−) Nutraceutical Composition I (F4V formulation) in supplement of M18 diet followed by immunization with Cholera Toxin (CT)/OVAlbumin (OVA).

FIG. 2A and FIG. 2B show the results from Study 1 and Study 2 depicted in FIG. 1A and FIG. 1B, plotting fecal anti-CT IgA/Total IgA ratio (CT IgA ratio; Arbitrary Units) in mice receiving microbiota from different undernourished Donors.

FIG. 2C shows the anti-CT IgG responses measured by ELISA in serum obtained from Study 2.

FIG. 3A results of linear mixed-effects models showing the effects of microbiota and dietary supplementation on the CT-IgA ratio in feces from all mice colonized with the R and HypoR microbiota identified in Studies 1 and 2. *, P<0.05.

FIG. 3B shows the percentage of CD38^{lo} GL7⁺ cells among CD19⁺TCRb⁻ cells in mesenteric lymph nodes (MLN) in mice in Studies 1 and 2 that were colonized with the R or HypoR microbiota and fed the M18 base diet or M18 supplemented with the Probiotic (−) Nutraceutical Composition I. P<0.05.

FIG. 3C is a heatmap showing the percent relative abundances of ASVs in mice colonized with either the R or HypoR community at 9, 15, 27, and 36 days post gavage.

FIG. 3D shows BugFACS analysis of feces obtained from R-colonized mice.

FIG. 4A is the experimental design of the co-housing study (Study 3). Four groups of 12 mice were dually-caged and fed the M18 diet. On day 3, two groups were colonized with the HypoR microbiota and two with the R microbiota. On day 10, one group harboring each microbiota was switched to the nutraceutical-supplemented M18 diet. On day 14, half the mice from each group were moved to a new, empty isolator, where they were each co-housed with another mouse as indicated.

FIG. 4B shows that mice fed the supplemented M18 diet exhibited increased CT-IgA ratios in their feces, and increased percentages of CD38loGL7+ cells among CD19+TCR-b− cells relative to mice fed the M18 diet. *, P<0.05, **, P<0.01, ***, P<0.001.

FIG. 4C is a heatmap showing the mean percent abundances of ASVs at 10, 13, 17, 28, and 40 days post-gavage in mice exposed to only the R microbiota, only the HypoR microbiota, or (through co-housing) the HypoR microbiota followed by the R microbiota (HypoR^{Ch-R}), or the R microbiota followed by HypoR microbiota (R^{Ch-HypoR}) in both the unsupplemented and nutraceutical supplemented M18 diet contexts. The column “Assigned microbiota” identifies ASVs previously demonstrated to be significantly associated with either the R or HypoR microbiota by indicator species analysis. The “Invade HypoR” and “Invade R” columns identify ASVs defined as invaders in the R->HypoR and HypoR->R directions, respectively. Only ASVs with a mean relative abundance of at least 1% in one mouse group on any day are included in the heatmap.

FIG. 4D Mice exposed to the R microbiota exhibited higher cecal concentrations of propionate than mice only exposed to HypoR microbiota. *, P<0.05,**, P<0.01, ***, P<0.001.

FIG. 4E Mice exposed to the R microbiota exhibited higher cecal concentrations of butyrate than mice only exposed to HypoR microbiota. *, P<0.05,**, P<0.01, ***, P<0.001.

FIG. 4F Mice exposed to the R microbiota exhibited higher cecal concentrations of succinate than mice only exposed to HypoR microbiota. *, P<0.05,***, P<0.001.

FIG. 5 shows the design of Study 4 (probiotic gavage), designed to study the effects of introducing (i) the intact R community, (ii) a 5-member consortium of cultured bacterial strains from the R community (Bacteroides acidifaciens, Bacteroides fragilis, Clostridioides difficile, Clostridium innocuum, Fusobacterium mortiferum), or (iii) Lactobacillus Rhamnosum GG (LGG) into the HypoR community.

FIG. 6 shows fecal anti-CT IgA/Total IgA data 7 d after the last CT vaccination from mice in Study 4 that were first colonized with the HypoR community and then gavaged with either R microbiota, the cultured 5 member R consortium or the Lactobacillus rhamnosus GG strain (LGG).

FIG. 7A shows the levels of activated memory B cells in the mesenteric lymph nodes in mice in Study 4.

FIG. 7B shows the levels of activated memory B cells in the spleens of mice in Study 4.

FIG. 8 shows the results 16S rDNA analysis of fecal samples collected from mice at the end of Study 4 showing the bacterial strains from the 5 member R cultured consortium (5memRCC) that were able to efficiently colonize mice previously gavaged with the HypoR microbiota.

FIG. 9 shows a heatmap of the bacterial taxa (Amplicon Sequence Variants; ASVs) that successfully invaded either the HypoR or R communities in the Study 3 (co-housing) and Study 4 (probiotic/direct gavage) experiments.

FIG. 10 shows ratios of fecal CT-specific IgA to total IgA in mice that were first colonized with the HypoR community and then gavaged with either the intact R microbiota or the cultured 5 member R culture consortium (5memRCC). *, P<0.05, **, P<0.01.

FIG. 11A,B show that HypoR mice exposed to the intact R microbiota or the 5 member R culture consortium (5memRCC) have higher concentrations of butyrate and propionate in their cecal contents than HypoR controls. *, P<0.05, ***, P<0.001.

FIG. 12A-C show features in the cecal metabolome identified by untargeted LC-Q-TOF MS that are associated with vaccine responsiveness in mice colonized with either the intact R community or the cultured 5 member R culture consortium (5memRCC). (A) m/z 144.1002 identified by collision-induced dissociation (CID) as proline betaine. (B) m/z 235.1078 and (C) its CID mass spectrum.

FIG. 13A-D show the results of non-targeted LC-QTOF MS of cecal contents obtained from HypoR mice subsequently colonized with the intact R community or the 5memRCC. (A) Principal components analysis of nontargeted metabolomic profiles separates the HypoR, HypoR+5memRCC and HypoR+R treatment groups. (B) Heatmap showing the concentrations of the top 10% of analytes with the strongest significant positive correlations with the CT-IgA ratio. (C) Heatmap showing the concentrations of analytes that differ between R^{Ch-R}, R^{Ch-HypoR} or HypoR^{Ch-R} mice fed the M18 diet or the nutraceutical-supplemented M18 diet in the cohousing experiment described in Study 3. (D) Heatmap showing the concentrations of analytes in the supernatants of in vitro cultures of the 5memRCC and its individual members, as well as uninoculated controls.

FIG. 14A and FIG. 14B show the effect of lipid-coated encapsulation on the color intensity of spirulina.

FIG. 15 Molecular mimicry concept. Microbiota-derived crossreactive antigens may act to prime T cells and/or may act to prime B cells.

### DETAILED DESCRIPTION OF THE INVENTION

The present invention relates to a nutraceutical composition capable of increasing an immune response to an antigen or antigenic composition (i.e. a vaccine) in a mammal, the nutraceutical composition comprising a probiotic, spirulina, a cereal and a micronutrient. One embodiment provides a nutraceutical composition for improving a person's health by addressing malnutrition and boosting the immune system and improving vaccine effectiveness in vulnerable populations in Least Developed Countries (LDCs), including undernourished children, lactating and pregnant mothers, and the elderly population suffering from immunosenescence and the immunocompromised patients in industrialized countries. One embodiment provides a nutraceutical formulation that comprises a combined prebiotic and probiotic (together a synbiotic) formulation, wherein the nutraceutial formulation, when administered to a mammal, improves immune response in a mammal to an antigen, e.g. a vaccine.

An embodiment provides a nutraceutical composition comprising microbiota capable of conferring an augmented CT-IgA response to an antigen or antigenic composition (i.e. a vaccine) in a mammal and further comprises spirulina, amaranth, flaxseed and micronutrients.

In an embodiment of the invention there is provided a nutraceutical composition comprising a probiotic species selected from Bacillus sp., Bifidobacterium sp., Enterococcus sp., Lactobacillus sp., Lactococcus sp., Propionibacterium sp., Pediococcus sp., Saccharomyces sp. and Streptococcus spa Bacteroides, a Clostridioides, a Clostridium, an Erysipelotrichaceae, a Firmicutes, a Flavonifractor, a Fusobacterium, a Lactobacillus, a Parabacteroides, a Peptoclostridrium, a Robinsoniella, or a Subdoligranulum species; spirulina, a cereal and micronutrients.

One embodiment of the invention provides a nutraceutical composition comprising a probiotic species selected from a Bacteroides, a Clostridioides, a Clostridium, an Erysipelotrichaceae, a Firmicutes, a Flavonifractor, a Fusobacterium, a Lactobacillus, a Parabacteroides, a Peptoclostridrium, a Robinsoniella, or a Subdoligranulum species; spirulina, a cereal and micronutrients.

One embodiment provides a nutraceutical composition comprising a probiotic species selected from a Bacteroides acidifaciens species, a Bacteroides fragilis species, a Clostridioides difficile species, a Clostridium innocuum species and a Fusobacterium mortiferum species; spirulina, a cereal and micronutrients. In certain embodiments, the microbiota C. difficile species does not possess genes encoding two principal glucosylating exotoxins, TcdA and TcdB.

Immune response to vaccines and natural antigens is often less robust in malnourished and vulnerable populations compared to well-nourished and healthy populations (Madhi, N Eng J Med 2010; Vesikary, Lancet 2007 and Bhandari, Lancet 2014). Thus, there is a need to develop a nutrient supplement/nutraceutical composition comprising microencapsulated prebiotic and/or probiotic (together being a ‘synbiotic’) wherein encapsulation may improve stability, encapsulated spirulina, cereals, and micronutrients comprising vitamins and minerals, such that the composition i) enhances or boosts the immune system and response to vaccines and/or natural antigens, particularly during the first 1000 days of an undernourished child's life; and ii) enhances or boosts the immune development and response to vaccines and/or natural antigens in vulnerable populations such as immunocompromised humans, pregnant females, lactating females and elderly humans.

Spirulina, which in its natural state is very green, also has an odor and a distinct taste. It is less palatable for humans in this form, so there is a need for encapsulated spirulina in a nutrient formulation capable of masking the undesirable flavour, odor and bright green color in order to obtain a galenic formulation more adapted for pediatric population. Therefore there is a need to provide encapsulated or coated spirulina and probiotic for use in a nutraceutical composition such that encapsulation does not negatively impact the nutritional value of the spirulina and probiotic present in the nutraceutial composition. In an embodiment of the invention, the spirulina present in the nutraceutical composition is spray-dried with a lipid formulation to coat or encapsulate the spirulina.

In embodiments of the invention, the coating or encapsulation materials are generally recognized as safe (GRAS), i.e. ingredients that can be used in food applications. In an embodiment, the lipids used to coat the spirulina are accepted as food additives, have a melting temperature higher than 40° C., and optionally have immunostimulating properties. In certain embodiments, the spray-dried spirulina coating comprises beta glucan, Microencapsulation materials may be applied using different microencapsulation techniques as described (Sultana, G. et al., “Encapsulation of probiotic bacteria with alginate-starch and evaluation of survival in simulated gastrointestinal conditions and in yoghurt” Int J Food Microbiol, 62 (2000), pp. 47-55; Muthukumarasamy et al., “Stability of Lactobacillus reuteri in different types of microcapsules” J Food Sci, 71 (2006), pp. 20-24; Anal and Singh “Recent advances in microencapsulation of probiotics for industrial applications and targeted delivery” Trends Food Sci Technol, 18 (2007), pp. 240-251; Ding and Shah “Effect of various encapsulating materials on the stability of probiotic bacteria” J Food Sci, 74 (2009), pp. M100-M107; Mokarram et al. “The influence of multistage alginate coating on survivability of potential probiotic bacteria in simulated gastric and intestinal juice” Food Res Int, 42 (2009), pp. 1040-1045; Chavarri et al. 2010; Cook et al. 2011, 2013). Spirulina may be coated by spray-drying with lipids to form a lipid-coated spray-dried spirulina. In certain embodiments, the spray-dried lipid-coated spirulina comprises a decreased color and or odor. In an embodiment, the spirulina may be spray-dried with plurol Oleique 497. In certain embodiments, the color of spirulina may be controlled through a higher lipid concentration, which decreases the green color of spirulina. In certain embodiments, the lipid used to prepare spray-dried spirulina comprises di- and tri-glyceride esters of fatty acids having a melting temperature of 40° C. or above. In certain embodiments, the lipid comprises Compritol 888 ATO (glyceryl dibehenate). In certain embodiments, the lipid comprises Gelucire 43/01. In certain In certain embodiments, the compritol 888 ATO has a melting range of 65-77° C. In certain embodiments, the Gelucire 43/01 has a melting temperature of 42° C.-46° C. In certain embodiments, the spray-dried spirulina coating comprises beta glucan, In certain embodiments, the spray-dried spirulina comprises ratio of 50% spirulina/50% lipid was kept for a better color and taste masking of spirulina.

In addition, certain embodiments provide the use of encapsulation of probiotic bacteria in milk proteins such as casein (Oliveira et al. “Stability of microencapsulated B. lactis (BI 01) and L. acidophilus (LAC 4) by complex coacervation followed by spray drying” J Microencapsul, 24 (2007), pp. 673-681; Heidebach et al. a “Microencapsulation of probiotic cells by means of rennet-gelation of milk proteins” Food Hydrocoll, 23 (2009), pp. 1670-1677 and Heidebach et al. “Transglutaminase-induced caseinate gelation for the microencapsulation of probiotic cells” Int Dairy J, 19 (2009), pp. 77-84), and whey protein (Doherty et al. “Survival of entrapped Lactobacillus rhamnosus GG in whey protein micro-beads during simulated ex vivo gastro-intestinal transit” Int Dairy J, 22 (2012), pp. 31-43).

Certain embodiments provide microencapsulation of probiotic bacteria in a natural polymer. Certain embodiments provide microencapsulation of probiotic species in alginate. Certain embodiments provide microencapsulation of probiotic bacteria in a pH-sensitive material (see Allan-Wojtas et al. “Microstructural studies of probiotic bacteria-loaded alginate microcapsules using standard electron microscopy techniques and anhydrous fixation” LWT Food Sci Technol, 41 (2008), pp. 101-108).

To fulfill many demands of a successful probiotic bacteria microencapsulation, different polymers may be used, natural and synthetic, to increase the resistance of such probiotic species against gastric conditions.

One embodiment of the invention provides a method of enhancing an immune response to a vaccine or antigen in a human, the method comprising administering an immune enhancing effective amount of a nutraceutical composition comprising: a probiotic species selected from Lactobacillus rhamnosus, Lactobacillus acidophilus; spirulina; cereal comprising flaxseed and amaranth; and micronutrients comprising i) vitamins B3, B6, C, D3, E and B9 and ii) minerals comprising magnesium, selenium and zinc; such that an enhanced immune response to a vaccine or antigen is observed in the human, as measured by an increase in mucosal vaccine specific IgA titer or as measured by an increase in systemic IgG titer.

One embodiment of the invention provides a method of using a probiotic to enhance the immune system in a human, the method comprising administering an immune enhancing effective amount of a probiotic species, wherein the probiotic species is a Bacteroides species, a Clostridium species, an Erysipelotrichaceae species, a Firmicutes species, a Flavonifractor species, a Fusobacterium species, a Lactobacillus species, a Parabacteroides species, a Peptoclostridrium species, a Robinsoniella species, a Subdoligranulum species or a combination thereof, such that the enhanced immune system is observable by an increased response to a vaccine or antigen in the human, as measured by an increase in mucosal vaccine specific IgA titer or as measured by an increase in systemic IgG titer.

One embodiment of the invention provides use of a nutraceutical composition as described herein for enhancing the immune system in a human by increasing an immune response to an antigen or a vaccine in said human, as measured by an increase in mucosal vaccine specific IgA titer or as measured by an increase in systemic IgG titer.

Embodiments of the invention relate to methods for improving an immune response and/or an IgA (CT-IgA) response in a mammal to an antigen or an antigenic composition (i.e. a vaccine), the method comprising administering a nutraceutical composition capable of increasing an immune response in the mammal, wherein the nutraceutical composition comprises a prebiotic, a probiotic or a synbiotic; spirulina; a cereal; and a micronutrient, such that an improved immune response and/or increase in vaccine specific IgA is observed.

One embodiment provides a method for increasing an immune response to an antigen or an antigenic composition such as a vaccine in a mammal, wherein the increased immune response may include an increase in germinal center memory B-cells in mesenteric lymph nodes, wherein the method comprising administering a nutraceutical composition capable of increasing an immune response as described herein to the mammal, wherein the increased immune response and/or increase in germinal center memory B-cells may be accompanied by changes in the cecal metabolome, including elevated short chain fatty acids.

One embodiment provides a method for increasing an immune response to an antigenic composition (e.g. an oral cholera toxin vaccine) wherein the method comprises administering to the mammal a nutraceutical composition capable of conferring augmented CT-IgA responses in the mammal.

One embodiment provides a nutraceutical composition comprising a prebiotic, spirulina and micronutrients. In particular embodiments, the prebiotic comprises a cereal, more specifically the prebiotic comprises flaxseed, amaranth, rice, oats, teff, bran, barley, wheat, rye, maize, millet, buckwheat, spelt, chia, quinoa or any other grain. In particular embodiments, the micronutrients comprise a vitamin and mineral. Particular embodiments provide a nutraceutical composition comprising a prebiotic, spirulina, micronutrients and a probiotic species. In particular embodiments, the probiotic comprises a Bacteroides species, a Fusobacterium species, a Clostridioides species, a Clostridium species, or a combination thereof. In particular embodiments, the probiotic comprises a Bacteroides acidifaciens species, a Bacteroides fragilis species, a non-toxigenic Clostridioides difficile species, a Clostridium innocuum species, a Fusobacterium mortiferum species or a combination thereof. In particular embodiments, the probiotic species is Fusobacterium mortiferum strain 9G6, Bacteroides acidifaciens strain 9G3, Bacteroides fragilis strain 8E3, Clostridium innocuum strain 9H7, Clostridioides difficile strain 9C4 or a combination thereof. In particular embodiments, the probiotic comprises a Bacillus sp., Bifidobacterium sp., Enterococcus sp., Lactobacillus sp., Lactococcus sp., Propionibacterium sp., Pediococcus sp., Saccharomyces sp. and Streptococcus spa Bacteroides, a Clostridioides, a Clostridium, an Erysipelotrichaceae, a Firmicutes, a Flavonifractor, a Fusobacterium, a Lactobacillus, a Parabacteroides, a Peptoclostridrium, a Robinsoniella, a Subdoligranulum species, or a combination thereof.

In particular embodiments, there is provided a nutraceutical composition comprising a probiotic; spirulina; a cereal; and micronutrients, wherein the probiotic comprises a probiotic selected from a Bacteroides species, a Clostridioides species, a Clostridium species, an Erysipelotrichaceae species, a Firmicutes species, a Flavonifractor species, a Fusobacterium species, a Lactobacillus species, a Parabacteroides species, a Peptoclostridrium species, a Robinsoniella species, a Subdoligranulum species, or a combination thereof. In particular embodiments, the probiotic comprises a Bacillus sp., Bifidobacterium sp., Enterococcus sp., Lactobacillus sp., Lactococcus sp., Propionibacterium sp., Pediococcus sp., Saccharomyces sp., a Streptococcus sp., or a combination thereof. In particular embodiments the probiotic comprises a Bacteroides species, a Clostridioides species, a Clostridium species, an Erysipelotrichaceae species, a Firmicutes species, a Flavonifractor species, a Fusobacterium species, a Lactobacillus species, a Parabacteroides species, a Peptoclostridrium species, a Robinsoniella species, a Subdoligranulum species or a combination thereof. In particular embodiments, the probiotic species comprises a Bacteroides species, a Fusobacterium species, a Clostridioides species, a Clostridium species, or a combination thereof. In particular embodiments, the probiotic species comprises a Bacteroides acidifaciens species, a Bacteroides fragilis species, a non-toxigenic Clostridioides difficile species, a Clostridium innocuum species, a Fusobacterium mortiferum species or a combination thereof. In particular embodiments, the probiotic species is Fusobacterium mortiferum strain 9G6, Bacteroides acidifaciens strain 9G3, Bacteroides fragilis strain 8E3, Clostridium innocuum strain 9H7, Clostridioides difficile strain 9C4 or a combination thereof.

Certain embodiments described herein provide a nutraceutical composition comprising a probiotic comprises a Bacillus sp., a Bifidobacterium sp., a Enterococcus sp., a Lactobacillus sp., a Lactococcus sp., a Propionibacterium sp., a Pediococcus sp., Saccharomyces sp., and a Streptococcus sp., Particular embodiments provide a nutraceutical composition wherein the probiotic comprises a Bacteroides species, a Clostridioides species, a Clostridium species, an Erysipelotrichaceae species, a Firmicutes species, a Flavonifractor species, a Fusobacterium species, a Lactobacillus species, a Parabacteroides, a Peptoclostridrium, a Robinsoniella, or a Subdoligranulum species. Particular embodiments provide a nutraceutical composition comprising a probiotic, wherein the probiotic comprises a Bacteroides species, a Clostridium species, an Erysipelotrichaceae species, a Firmicutes species, a Flavonifractor species, a Fusobacterium species, a Lactobacillus species, a Parabacteroides, a Clostridioides species; a Peptoclostridrium, a Robinsoniella, or a Subdoligranulum species. In particular embodiments, the probiotic comprises a Bacteroides species, a Fusobacterium species, a Parabacteroides, species a Clostridioides species, or a Clostridium species or a combination thereof. In particular embodiments, the probiotic comprises a Bacteroides acidifaciens species, a Bacteroides fragilis species, a non-toxigenic Clostridioides difficile species, a Clostridium innocuum species, Fusobacterium mortiferum species or a combination thereof. In particular embodiments, the probiotic comprises Fusobacterium mortiferum strain 9G6, Bacteroides acidifaciens strain 9G3, Bacteroides fragilis strain 8E3, Clostridium innocuum strain 9H7, Clostridioides difficile strain 9C4 or a combination thereof.

In a particular embodiment, the nutraceutical composition comprises a composition as described herein, comprising a non-toxigenic Clostridioides species. In particular embodiments, the non-toxigenic Clostridioides difficile species does not possess genes encoding glucosylating exotoxins TcdA and TcdB.

In a particular embodiment, a nutraceutical composition as described herein comprises a cereal, wherein the cereal comprises flaxseed, amaranth, rice, oats, teff, bran, barley, wheat, rye, maize, millet, buckwheat, spelt, chia, quinoa or any other grain. In a particular embodiment, a nutraceutical composition as described herein comprises a cereal, wherein the cereal comprises flaxseed and amaranth. In a particular embodiment, a nutraceutical composition as described herein comprises micronutrients, wherein the micronutrients comprise a vitamin and mineral. In a particular embodiment of a nutraceutical composition that comprises micronutrients as described herein, the micronutrients comprise i) a vitamin A (α-carotene, β-carotene, retinol), a vitamin B1 (thiamin), a vitamin B2 (riboflavin), a vitamin B3 (niacin), a vitamin B5 (pantothenic acid), a vitamin B6 (pyroxidine), a vitamin B7 (biotin), a vitamin B9 (folate or folic acid), a vitamin B12 (cobalamin or cyanocobalamin), a vitamin C (ascorbic acid or ascorbate), a vitamin D1 (a mixture of lumisterol and califerol), a vitamin D2 (ergocalciferol), a vitamin D3 (cholecalciferol), a vitamin E (α-tocopherol) or a vitamin K (phytonadione), and ii) a mineral comprising calcium, chloride, chromium, copper, iodine, iron, magnesium, manganese, molybdenum, phosphorus, potassium, selenium, sodium or zinc. In a particular embodiment of a nutraceutical composition that comprises micronutrients as described herein, the micronutrients comprise vitamin B3, vitamin B6, vitamin C, vitamin D3, vitamin E or vitamin B9. In a particular embodiment of a nutraceutical composition that comprises micronutrients as described herein, the micronutrients comprise magnesium, selenium or zinc.

In a particular embodiment, a nutraceutical composition as described herein comprises a spirulina, wherein the spirulina is encapsulated. In particular embodiments, the encapsulated spirulina is encapsulated with a lipid carrier emulsion. In particular embodiments, the lipid carrier emulsion comprises a nonionic emulsifier. In particular embodiments, the nonionic emulsifier comprises an oleic acid, a dibehenate, a di- and triglyceride ester of a fatty acid, a beta-glucan, or a combination thereof. In particular embodiments, the lipid carrier emulsion comprises a microemulsion. In particular embodiments, the lipid carrier microemulsion comprises an oleic acid. In particular embodiments, the lipid carrier microemulsion comprises a dibehenate. In particular embodiments, the lipid carrier microemulsion comprises a di- or tri-glyceride. In particular embodiments, the lipid carrier microemulsion comprises a beta-glucan. In particular embodiments, the lipid carrier microemulsion comprises Plurol® Oleique CC 497 CG, Compritol 888 ATO, Gelucire 43/01.

In a particular embodiment as described herein, the lipid carrier microemulsion is formulated with the spirulina at a concentration of up to 1%, 5%, 10% 15%, 20%, 30% 40% or 50% (by weight).

In a particular embodiment, when a nutraceutical as described herein comprises a spirulina, a cereal, and micronutrients, the spirulina is present in an amount of 5-15% (dry weight), the cereal comprises flaxseed and amaranth, and the flaxseed is present in an amount of 1-5% (dry weight), the amaranth is present in an amount of 5-15% (dry weight) and the micronutrients are present in an amount of 0.02-0.05% (dry weight). In a particular embodiment, the spirulina is present in an amount of 5% (dry weight), the flaxseed is present in an amount of 2.5% (dry weight), the amaranth is present in an amount of 10% (dry weight) and the micronutrients are present in an amount of 0.025% (dry weight).

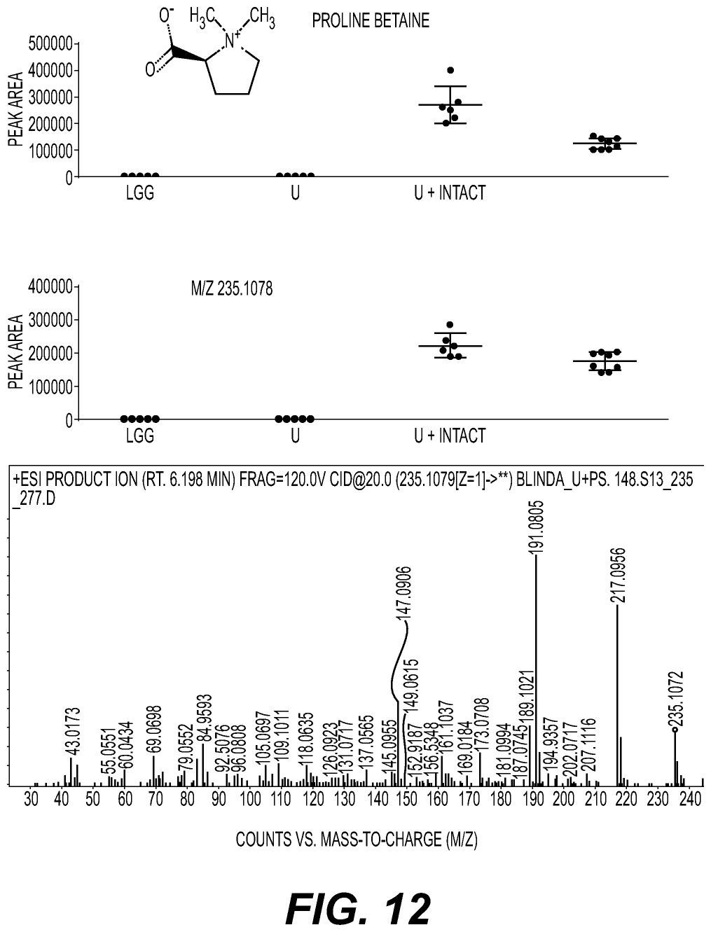

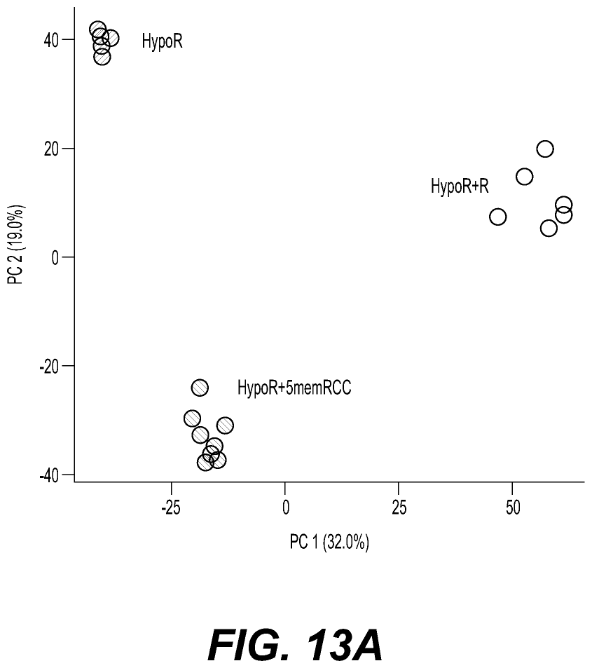

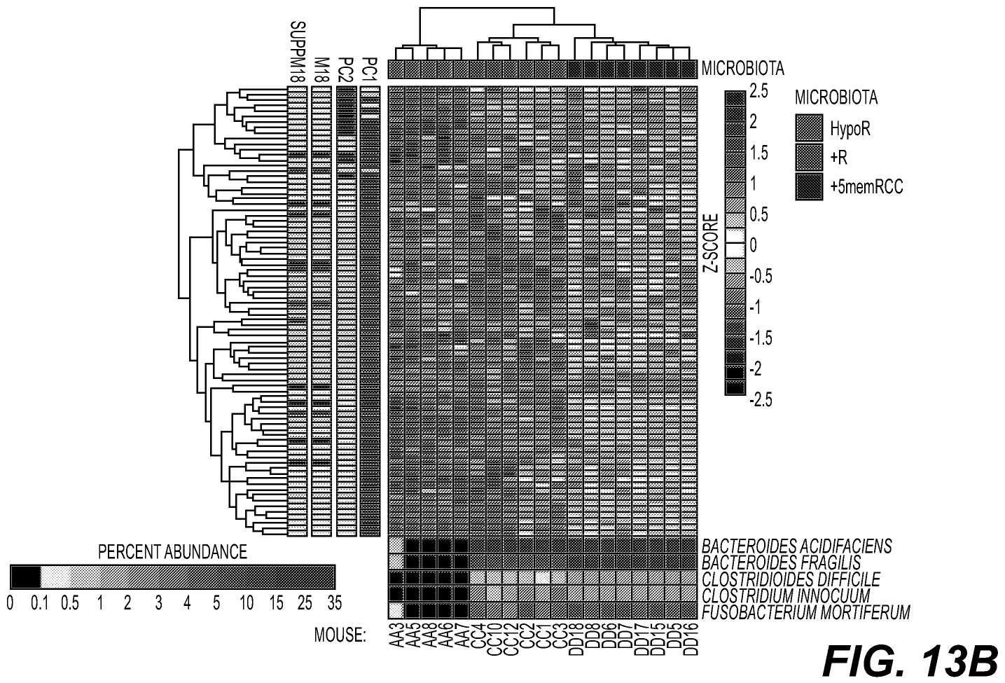

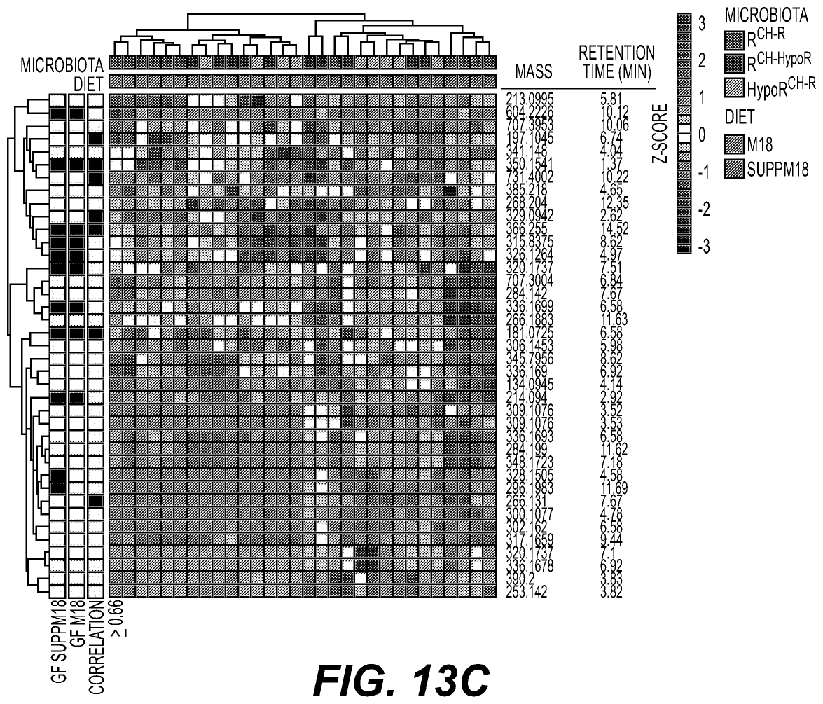

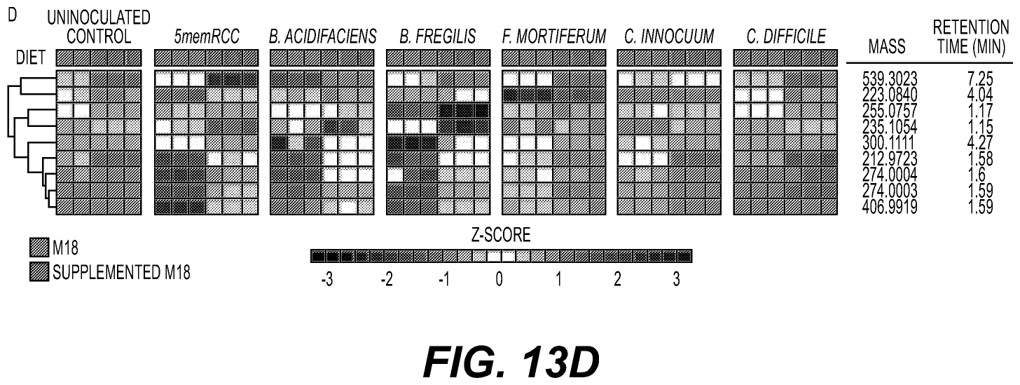

In a particular embodiment, a nutraceutical composition as described herein comprises a probiotic, wherein the probiotic is encapsulated. In a particular embodiment, the encapsulated probiotic is microencapsulated. In a particular embodiment, a nutraceutical composition as described herein comprises a microbiota metabolite, including a metabolite as set forth in any of FIG. 12A, FIG. 12B, 12C and/or FIG. 13C, or a combination thereof. In a particular embodiment, a nutraceutical composition as described herein comprises an an adjuvant. In a particular embodiment, the adjuvant is microbiota metabolite. In a particular embodiment, the microbiota metabolite comprises a metabolite as set forth in any of FIG. 12A, FIG. 12B, 12C and/or FIG. 13C, or a combination thereof.

One embodiment of the invention provides a method of enhancing an immune response to a vaccine or antigen in a human, the method comprising administering an immune enhancing effective amount of a nutraceutical composition as described herein. In a particular embodiment, the method of enhancing an immune response to a vaccine or antigen in a human comprises administering a nutraceutical composition as described herein comprising a probiotic. In particular embodiments, the probiotic comprises a Lactobacillus rhamnosus species, Lactobacillus acidophilus species;

In a particular embodiment, the method of enhancing an immune response to a vaccine or antigen in a human comprises administering a nutraceutical composition as described herein comprising a spirulina. In a particular embodiment, the method of enhancing an immune response to a vaccine or antigen in a human comprises administering a nutraceutical composition as described herein comprising a cereal. In particular embodiments, the cereal comprises flaxseed and amaranth. In a particular embodiment, the method of enhancing an immune response to a vaccine or antigen in a human comprises administering a nutraceutical composition as described herein comprising micronutrients and minerals. In particular embodiments, the micronutrients comprises vitamin B3, vitamin B6, vitamin C, vitamin D3, vitamin E and vitamin B9; and the minerals comprise magnesium, selenium and zinc. In a particular embodiment, the method of enhancing an immune response to a vaccine or antigen in a human comprises administering a nutraceutical composition as described herein such that an enhanced immune response to a vaccine or antigen is observed in the human, as measured by an increase in mucosal IgA titer or as measured by an increase in systemic IgG titer. In particular embodiments, the human is selected from a child, a female, an elderly human, an immunocompromised human or an immunosenescent human. In particular embodiments, the human is a child and more particularly, the child is an undernourished child. In particular embodiments, the human is a female, more particularly the female is a pregnant female. In a particular embodiment, the female is a lactating female. In particular embodiments, the human is an elderly human. In particular embodiments, the human is an immunocompromised human, more particularly, the immunocompromised human has been diagnosed with cancer, or is infected with a virus including human immunodeficiency virus (HIV), hepatitis B virus (HBV), hepatitis D virus (HDV), hepatitis C virus (HCV), Dengue virus, Influenza virus, Zika virus, or Epstein-barr virus.

In a particular embodiment of a nutraceutical composition as described herein, the nutraceutical composition is formulated as a powder, a tablet, a capsule, a bead, a gel, a paste, or a liquid. In a particular embodiment, the nutraceutical composition is formulated as a powder that is reconstituted in liquid, more particularly, the powder is reconstituted in up to 50, 75, 100, 150, 200, 250, 300 or 400 mL of water.

In a particular embodiment of a nutraceutical composition as described herein, the nutraceutical composition is provided at a dose of up to 10, 20, 30, 40, 50 or 100 g/kg body weight, more preferably at a dose of up to 0.5, 1, 2, 3, 4, 5 or 10 g/kg body weight. In a particular embodiment of a nutraceutical composition as described herein the nutraceutical composition is administered orally.

In a particular embodiment of a nutraceutical composition as described herein, the nutraceutical composition is administered 1× to 3× daily, or is administered 1× to 6× weekly, or is administered biweekly, or is administered monthly. In a particular embodiment of a nutraceutical composition as described herein, the nutraceutical composition is administered 1, 2, 3, 4, 5 or 6 months prior to an immunization. In a particular embodiment of a nutraceutical as described herein, the nutraceutical composition is administered 1, 2, 3 4, 5 or 6 months post-immunization. In a particular embodiment of a nutraceutical as described herein, the nutraceutical composition is further administered 1, 2, 5, 10, 20 or 30 years post-immunization. In a particular embodiment of a nutraceutical as described herein, the nutraceutical composition is administered over the lifetime of the human.

In a particular embodiment there is provided a method of enhancing an immune response to a vaccine or antigen in a human, the method comprising administering a nutraceutical composition as described herein, wherein the nutraceutical composition further comprises a metabolite as set forth in any of FIG. 12A, FIG. 12B, 12C and/or FIG. 13C, or a combination thereof.

In a particular embodiment there is provided a method of enhancing an immune response to a vaccine or antigen in a human, the method comprising administering an immune enhancing effective amount of a probiotic, wherein the probiotic comprises a Bacteroides species, a Clostridioides species, Clostridium species, an Erysipelotrichaceae species, a Firmicutes species, a Flavonifractor species, a Fusobacterium species, a Lactobacillus species, a Parabacteroides species, a Peptoclostridrium species, a Robinsoniella species, a Subdoligranulum species or a combination thereof, such that the enhanced immune system is observable by an increased response to a vaccine or antigen in the human, as measured by an increase in mucosal IgA titer or as measured by an increase in systemic IgG titer. In a particular embodiment, the method comprises administering an immune enhancing effective amount of a probiotic, wherein the probiotic comprises a Fusobacterium species, a Clostridioides species, a Clostridium species, or a combination thereof. In a particular embodiment, the method comprises administering an immune enhancing effective amount of a probiotic, wherein the probiotic comprises a Bacteroides acidifaciens species, a Bacteroides fragilis species, a non-toxigenic Clostridioides difficile species, a Clostridium innocuum species, a Fusobacterium mortiferum species or a combination thereof. In a particular embodiment, the method comprises administering an immune enhancing effective amount of a probiotic, wherein the probiotic comprises Fusobacterium mortiferum strain 9G6, Bacteroides acidifaciens strain 9G3, Bacteroides fragilis strain 8E3, Clostridium innocuum strain 9H7, Clostridioides difficile strain 9C4 or a combination thereof.

In a particular embodiment wherein there is provided a method of enhancing an immune response to a vaccine or antigen in a human, wherein the method comprises administering an immune enhancing effective amount of a probiotic, wherein the human is a child, a female, or an elderly human. In a particular embodiment there is provided a method of enhancing an immune response to a vaccine or antigen in a human, wherein the human is a child. In a particular embodiment, the child is an undernourished child. In a particular embodiment wherein there is provided a method of enhancing an immune response to a vaccine or antigen in a human, wherein the human is a female. In a particular embodiment, the female is a pregnant female. In a particular embodiment, the female is a lactating female. In a particular embodiment wherein there is provided a method of enhancing an immune response to a vaccine or antigen in a human, the human is an elderly human. In a particular embodiment, the human is an immunocompromised human. More particularly, the immunocompromised human has been diagnosed with cancer, or is infected with a virus including human immunodeficiency virus (HIV), hepatitis B virus (HBV), hepatitis D virus (HDV), hepatitis C virus (HCV), Dengue virus, Influenza virus, Zika virus, or Epstein-barr virus.

One particular embodiment provides use of a nutraceutical composition as described herein for enhancing the immune system by increasing an immune response to an antigen or a vaccine in a human, as measured by an increase in mucosal IgA titer or as measured by an increase in systemic IgG titer. In a particular embodiment there is provided use of a nutraceutical composition as described herein for enhancing the immune system by increasing an immune response to an antigen or a vaccine in a human, wherein the human is a child, a female, or an elderly human. In a particular embodiment of use of a nutraceutical composition as described herein for enhancing the immune system by increasing an immune response to an antigen or a vaccine in a human, the human is a child, and more particularly the child is an undernourished child. In a particular embodiment of use of a nutraceutical composition as described herein for enhancing the immune system by increasing an immune response to an antigen or a vaccine in a human, the human is a female. In particular embodiments, the female is a pregnant female. In particular embodiments, the female is a lactating female. In a particular embodiment of use of a nutraceutical composition as described herein for enhancing the immune system by increasing an immune response to an antigen or a vaccine in a human, the human is an elderly human. In a particular embodiment of use of a nutraceutical composition as described herein for enhancing the immune system by increasing an immune response to an antigen or a vaccine in a human, the human is an immunocompromised human; more particularly, the immunocompromised human has been diagnosed with cancer, or is infected with a virus including human immunodeficiency virus (HIV), hepatitis B virus (HBV), hepatitis D virus (HDV), hepatitis C virus (HCV), Dengue virus, Influenza virus, Zika virus, or Epstein-barr virus.

One embodiment of the invention provides use of a nutraceutical composition as described herein in the manufacture of a medicament for the enhancement of an immune response in a human. In a particular embodiment of a use of a nutraceutical composition as described herein in the manufacture of a medicament for the enhancement of an immune response in a human, the human is a child, a female, or an elderly human.

In a particular embodiment of a use of a nutraceutical composition as described herein in the manufacture of a medicament for the enhancement of an immune response in a human, the human is a child. In a particular embodiment, the child is an undernourished child. In a particular embodiment of a use of a nutraceutical composition as described herein in the manufacture of a medicament for the enhancement of an immune response in a human, the human is a female. In a particular embodiment, the female is a pregnant female. In a particular embodiment, the female is a lactating female. In a particular embodiment of a use of a nutraceutical composition as described herein in the manufacture of a medicament for the enhancement of an immune response in a human, the human is an elderly human. In a particular embodiment of a use of a nutraceutical composition as described herein in the manufacture of a medicament for the enhancement of an immune response in a human, the human is an immunocompromised human. In a particular embodiment, the immunocompromised human has been diagnosed with cancer, or is infected with a virus including human immunodeficiency virus (HIV), hepatitis B virus (HBV), hepatitis D virus (HDV), hepatitis C virus (HCV), Dengue virus, Influenza virus, Zika virus, or Epstein-barr virus.

### Definitions

“5memRCC” refers to the 5 member bacterial consortium cultured from a vaccine responsive (R) microbiota.

“ANOVA” as used herein stands for Analysis Of VAriance, a statistical method that divides variation in a set of observations into distinct components. It can be used to determine whether there are any statistically significant differences between the means of three of more independent (unrelated) groups. For example, the one-way ANOVA tests the null hypothesis:

$${H}_{0} : {μ}_{1} = {μ}_{2} = … = {μ}_{κ}$$

wherein μ=group mean and κ=number of groups, and if this analysis provides a statistically significant result, it indicates that at least two group means are statistically different from each other.

“Anthropometric” as used herein means measurements or scientific study of measurements and proportions of the human body. Examples include height, weight, body mass index, mid-upper arm circumference (MUAC), triceps skin fold (TSF) circumference, mid-are muscle circumference, head circumference and the like.

“ASV” as used herein means amplicon sequence variant.

“Base diet” as used herein means the Mirpur-18 (M18) diet set forth in Table 2A, or any of the base diet variations described herein, by component and/or by weight, of the M18 diet of Table 2A.

“Cereal” as used herein means the edible component or grain portion of any grass.

CT=Cholera Toxin (CT)

CTB=the B subunit of cholera toxin

d=day or days

“Encapsulation” or “encapsulated” as used herein means to coat, encompass, surround, associate or adhere one substance around or to another substance being encapsulated. Substances that may be encapsulated include the probiotics, spirulina or any other component of the nutraceutical compositions describe herein.

“F4V” as used herein means nutraceutical compositions/supplements. F4V, as used in the present application and figures, and is synonymous with Probiotic (−) Nutraceutical Composition I, or just Nutraceutical Composition I.

“HAZ” as used herein refers to World Health Organization (WHO) recommended use of height-for-age Z scores (WHZ) to assess malnutrition prevalence in children.

“Mirpur-18 base diet”, first described by Gehrig et al. “Effects of microbiota-directed foods in gnobiotic animals and undernourished children”, Science 365, 6449 and referred to herein, is also referred to as Mirpur-18 diet or M18 diet or M18, and is described herein in Table 2A.

“Supplemented Mirpur-18 diet”, also referred to supplemented M18 diet, supplemented M18 or Nutraceutical Composition I-supplemented M18 diet is described herein in Table 2B.

“An immune enhancing effective amount of a nutraceutical composition” as used herein means an amount of nutraceutical composition sufficient to provoke an enhanced immune response in a subject, including a human, to an antigen or to a vaccine, as measured by an increase in mucosal vaccine specific IgA titer or as measured by an increase in systemic IgG titer.

“LDC” or “LDCs” as used herein means Least Developed Countries. Least Developed Countries includes, but is not limited, to the UN list of Least Developed Countries (updated March 2018) which includes Afghanistan, Angola, Bangladesh, Benin, Bhutan, Burkina Faso, Burundi, Cambodia, Central African Republic, Chad, Camoros, Democratic Replublic of Congo, Djobouti, Eritrea, Ethiopia, Gambia, Guinea, Guinea-Bissau, Haiti, Kiribati, Lao People's Democratic Republic, Lesotho, Liberia, Madagascar, Malawi, Mali, Mauritania, Mozambique, Myanmar, Nepal, Niger, Rwanda, Sao Tome and Principe, Senegal, Sierra Leone, Solomon Islands, Somalia, South Sudan, Sudan, Timor-Leste, Togo, Tuvalu, Uganda, United Republic of Tanzania, Vanuatu, Yemen, Zambia (https://www.un.org/development/desa/dpad/wp-content/uploads/sites/45/publication/Idc_list.pdf).

“Microbiome” as used herein means all of the genetic material that makes up or is contained in the human gut microbiota.

“Microbiota” as used herein refers to the entire collection of microorganisms in the human gut.

“Microemulsion” as used herein means a system wherein one phase, the dispersed phase (such as an oil or lipid), is solubilized in another phase, the continuous phase (such as water), to form a new component known as an emulsifier. Microemulsions form spontaneously, typically with formation of small droplets (micelles of the dispersed phase within the continuous phase) with diameters of 10-100 nm. Examples of lipids to be used in a microemulsion is Plurol® Oleique CC 497 CG (polyglycerol ester of oleic acid—CAS 9007-48-1), which is available from Gattefossé (https://www.ulprospectorcom/en/eu/PersonalCare/Detail/3983/113082/Plurol-Oleique-CC-497-CG), Compritol 888 ATO and Gelucire 43/01 which are also available from Gattefossé, and beta-glucans, which act as immunomodulator agents that can boost the immune system by stimulating activity of macrophages and lymphocytes. Thus, coating spirulina with beta-glucans may result in synergies.

“Micronutrient(s)” as used herein refers to a substance, often present in trace amounts, important or useful for the normal growth and development of an animal, including a human. A micronutrient may be a vitamin, mineral or compound, such as an amino acid, co-enzyme, fatty acid, or a neurotransmitter precursor such as choline. A micronutrient may be an element or compound not produced by the animal which relies on the micronutrient for normal growth, such as calcium or iron or zinc. A micronutrient may also be a compound or substance that is produced only in limited amounts by the animal which relies on it for normal growth.

MLN=mesenteric lymph nodule(s)

“MLN Treg(s)” as used herein refers to T regulatory cell(s) in mesenteric lymph node(s)

mo=month or months

“Non-toxigenic” as used herein refers to a bacterial species that does not possess genes encoding endotoxins which may mediate pathogenic effects. Specifically, by example, a non-toxigenic Clostridioides difficile species will not possess genes encoding the glucosylating exotoxins TcdA and Tcd B.

“Nutraceutical” as used herein refers to an edible additive for food or beverage such that the substance provides a medicinal, health or immunological benefit to an animal, including a human, that ingests the nutraceutical. The term nutraceutical originally was coined as a combination of the words “nutrition” and “pharmaceutical”. A nutraceutical may be a food/beverage or food/beverage component, such as a dietary supplement or a food additive. As used herein, the term food is intended to encompass any edible substance, which substance may be in solid, liquid, paste, tablet or other orally ingestible form. A nutraceutical may also supplement the diet, and includes traditional dietary supplements such as vitamins, minerals, herbs, oils and substances such as glucosamine, amino acids and other dietary supplements. As used herein, a nutraceutical is intended for oral ingestion and provides a health, medicinal or immunological benefit that may aid in disease prevention, disease treatment, and immunologic response. A nutraceutical may be a pharmaceutical-grade nutrient with standardized properties. A nutraceutical may also be a combination of substances including any combination of dietary supplements, food additives, vitamins, minerals, probiotics, prebiotics, spirulina, cereals and other substances that confer a health benefit to the animal, including a human, that ingests the nutraceutical. A nutraceutical may also be considered a functional food or functional food ingredient that provides a health, medicinal or immunological benefit in addition to the basic nutritional value of the food or food ingredient. A nutraceutical may be any functional or medicinal food that plays a role in maintaining well being, enhancing health, modulating immunity and thereby aiding in preventing as well as treating specific diseases.

“OTU(s)” as used herein means operational taxonomic unit(s)

“PERMANOVA” as used herein refers to PERmutational Multivariate ANalysis Of VAriance

“Prebiotic” as used herein refers to any substance that when consumed or administered provides non-digestible fibers that, when passed through the intestine and reach the colon, are fermented by microflora in the gut. Prebiotic(s) include cereals, dietary fiber, carbohydrates, polysaccharides and oligosaccharides, peptides and peptidocarbohydrates and peptidosaccharides. Prebiotics are fermented by beneficial bacteria in the gut as a source of fuel and to enhance gut flora health. Prebiotics can be considered to feed probiotics and gut microflora.

“Probiotic” as used herein refers to any substance(s) that when consumed or administered stimulates the growth of microorganisms in an animal, including a human, especially those bacteria with beneficial properties. Probiotics include live micro-organisms that confer a health benefit on the host. Examples of probiotics include the intestinal microorganism flora (microflora) of an animal, including a human, certain foods such as cheese and yogurt which contain beneficial bacteria, and dietary supplements such as powdered probiotic drink formulas or powdered probiotics available in capsules. Probiotics are capable of maintaining and/or restoring beneficial bacteria to the digestive tract.

sp.=species
“spirulina” as used herein means a filamentous cyanobacteria (microscopic blue-green algae) that form tangled masses in warm alkaline lakes in Africa and Central and South America. The two most commonly utilized species of spirulina are Arthrospira platensis and Arthrospira maxima.

“Synbiotic” as used herein means a combined prebiotic and probiotic.

TEER as used herein refers to Transepithelial Electrical Resistance.

“WAZ” as used herein refers to World Health Organization (WHO) recommended use of weight-for-age Z scores (WHZ) to assess malnutrition prevalence in children.

“WHZ” as used herein refers to World Health Organization (WHO) recommended use of weight-for-height Z scores (WHZ) to assess malnutrition prevalence in children.

Z score” as used herein refers to a malnutrition score using various measurements to arrive at the Z score. In statistics, a Z-score is also referred to as a standard score, and it provides an idea of how far from the mean a given data point is. A Z-score, when placed on a normal (standard) distribution curve will range from −3 standard deviations to +3 standard deviations from the mean. A Z-score can be determined if the mean (μ) and population standard deviation (σ) is known. The WHO Global Database on Child Growth and Malnutrition uses a Z-score cut-off of <−2 sd (standard deviation) from the mean weight-for-age, height-for-age and weight-for-height values in children to define low malnutrition; >−3 sd from mean weight-for-age, height-for-age and weight-for-height in children to define severe malnutrition; and >−2 sd and <−3 sd from weight-for-age, height-for-age and weight-for-height in children, to define moderate malnutrition in children.

Embodiments herein relating to “vaccine compositions” are also applicable to embodiments relating to “immunogenic compositions”, and vice versa, wherein each term may be used interchangeably to describe a composition which provides an immunostimulatory effect upon administration to an animal.

### Nutraceutical Compositions/Routes of Administration/Dosages

Compositions—General

In embodiments of the invention, nutraceutical compositions or supplements suitable for boosting an immune response, for example as a nutritive oral adjuvant for vaccination, will generally comprise spirulina, cereals, micronutrients, and may comprise probiotics or no probiotic. Spirulina may be coated or encapsulated. In certain embodiments, encapsulation or coating may help to mask color, odor and/or flavour. In embodiments, cereals may be selected from flaxseed, amaranth, teff, rice, oats, bran, barley, wheat, rye, maize, millet, buckwheat, spelt, chia, quinoa or any other grain. In embodiments of the invention, micronutrients include vitamins and minerals. In embodiments, vitamins may be selected from vitamin A (α-carotene, β-carotene, retinol), B1 (thiamin), B2 (riboflavin), B3 (niacin), B5 (pantothenic acid), B6 (pyroxidine), B7 (biotin), B9 (folate or folic acid), B12 (cobalamin or cyanocobalamin), C (ascorbic acid or ascorbate), D1 (a mixture of lumisterol and califerol), D2 (ergocalciferol), D3 (cholecalciferol), E (α-tocopherol) and K (phytonadione). In embodiments of the invention, minerals may be selected from calcium, chloride, chromium, copper, iodine, iron, magnesium, manganese, molybdenum, phosphorus, potassium, selenium, sodium and zinc.

Nutraceutical compositions may also comprise fish oil such as cod-liver oil; rapeseed or rapeseed oil; lipid compounds such as fatty acids including omega-3 and omega-6 fatty acids; phospholipids; ceramides; and sterols including phytosterols, zoosterols and mycosterols. Examples of phytosterols include avenosterol, β-sitosterol, campesterol and stigmasterol, as well as fully-saturated phytosterols known as phytostanols such as sitostanol and coprostanol. Examples of zoosterols include cholesterol, 24-isopropylcholesterol, 7-dehydrocholesterol, lanosterol, nicasterol, oxysterol, 4-methylcholestan-8(14), 24-diene-3β-ol, ganoderic acid and gorgosterol. Examples of mycosterols include ergosterol, antrosterol and saringosterol.

Examples of probiotic that may be added to any nutraceutical composition in embodiments herein include a Bacillus sp., a Bifidobacterium sp., an Enterococcus sp., a Lactobacillus sp., a Lactococcus sp., a Pediococcus sp., a Saccharomyces sp., a Streptococcus sp., a Bacteroides sp., a Clostridioides sp., a Clostridium sp., an Erysipelotrichaceae sp., a Firmicutes sp., a Flavonifractor sp., a Fusobacterium sp., a Lactobacillus sp., a Parabacteroides sp., a Peptoclostridrium sp., a Robinsoniella sp., or a Subdoligranulum species.

Examples of specific species include Bacillus coagulans, Bacillus laterosporus, Bifidobacterium breve, Bifidobacterium bifidum, Bifidobacterium infantis, Bifidobacterium lactis, Bifidobacterium longum, Enterococcus faecium, Lactobacillus acidophilus, Lactobacillus brevis, Lactobacillus bulgaricus, Lactobacillus casei, Lactobacillus fermentum, Lactobacillus gasseri, Lactobacillus helveticus, Lactobacillus infantis, Lactobacillus lactis, Lactobacillus plantarum, Lactobacillus paracasei, Lactobacillus rhamnosus, Lactobacillus salivarius, Lactobacillus reuteri, Lactococcus lactis, Pediococcus acidilactici, Saccharomyces boulardii, Streptococcus thermophilus Bacteroides acidifaciens, Bacteroides fragilis, Clostridioides difficile, Clostridium innocuum and Fusobacterium mortiferum. In certain embodiments, the probiotic species is Fusobacterium mortiferum strain 9G6, Bacteroides acidifaciens strain 9G3, Bacteroides fragilis strain 8E3, Clostridium innocuum strain 9H7, or Clostridioides difficile strain 9C4. Other examples of probiotics that may be added in embodiments herein to any nutraceutical composition described herein include new probiotics experimentally identified as having adjuvant properties for increasing an immune response to an antigen, referred to herein as a “probiotic adjuvant”, whether the antigen is encountered naturally or via vaccination with an immunogenic composition, e.g. a vaccine. In embodiments, an experimentally identified probiotic adjuvant may be identified by comparing immune response to the antigen in the presence of other probiotics, and selecting those probiotics that impart improved immune response to an antigen compared to other probiotic and/or compared to the immune response observed to the antigen without the adjuvant probiotic.

In certain embodiments, a nutraceutical composition may comprise spirulina, cereals, micronutrients, and may comprise probiotics or no probiotic. In certain embodiments, the spirulina in the composition may be coated or encapsulated to, e.g., help mask color, odor and/or flavour of the spirulina. In certain embodiments, the cereals of the nutraceutical composition may comprise cereals selected from flaxseed, amaranth, teff, rice, oats, bran, barley, wheat, rye, maize, millet, buckwheat, spelt, chia, quinoa or any other grain. In certain embodiments, the micronutrients of the nutraceutical composition include vitamins and minerals wherein the vitamins are selected from vitamin A (α-carotene, β-carotene, retinol), B1 (thiamin), B2 (riboflavin), B3 (niacin), B5 (pantothenic acid), B6 (pyroxidine), B7 (biotin), B9 (folate or folic acid), B12 (cobalamin or cyanocobalamin), C (ascorbic acid or ascorbate), D1 (a mixture of lumisterol and califerol), D2 (ergocalciferol), D3 (cholecalciferol), E (α-tocopherol) and K (phytonadione), and wherein the minerals are selected from calcium, chloride, chromium, copper, iodine, iron, magnesium, manganese, molybdenum, phosphorus, potassium, selenium, sodium and zinc.

In certain embodiments the nutraceutical compositions as described herein may optionally comprise: fish oil such as cod-liver oil; rapeseed or rapeseed oil; a lipid compound such as a fatty acid including an omega-3 or an omega-6 fatty acid; a phospholipid; a ceramide; or a sterol including a phytosterol, zoosterol or mycosterol. In certain embodiments the phytosterol is selected from β-sitosterol, campesterol and stigmasterol. In certain embodiments, the zoosterol is cholesterol. In certain embodiments, the mycosterol is ergosterol.

In certain embodiments, the nutraceutical composition optionally comprises a probiotic wherein the probiotic is selected from a Bacillus sp., a Bifidobacterium sp., an Enterococcus sp., a Lactobacillus sp., a Lactococcus sp., a Pediococcus sp., a Saccharomyces sp., or a Streptococcus sp. In certain embodiments, the nutraceutical composition comprises a Bacillus sp. selected from Bacillus coagulans and Bacillus laterosporus. In certain embodiments, the nutraceutical composition comprises a Bifidobacterium sp. selected from Bifidobacterium breve, Bifidobacterium bifidum, Bifidobacterium infantis, Bifidobacterium lactis and Bifidobacterium longum. In certain embodiments, the nutraceutical composition comprises an Enterococcus sp. that is Enterococcus faecium. In certain embodiments, the nutraceutical composition comprises a Lactobacillus sp. selected from Lactobacillus acidophilus, Lactobacillus brevis, Lactobacillus bulgaricus, Lactobacillus casei, Lactobacillus fermentum, Lactobacillus gasseri, Lactobacillus helveticus, Lactobacillus infantis, Lactobacillus lactis, Lactobacillus plantarum, Lactobacillus paracasei, Lactobacillus rhamnosus, Lactobacillus reuteri and Lactobacillus salivarius. In certain embodiments, the nutraceutical composition comprises a Lactococcus sp. that is Lactococcus lactis. In certain embodiments, the nutraceutical composition comprises a Pediococcus sp. that is Pediococcus acidilactici. In certain embodiments, the nutraceutical composition comprises a Saccharomyces sp. that is Saccharomyces boulardii. In certain embodiments, the nutraceutical composition comprises a Streptococcus sp. that is and Streptococcus thermophilus. In particular embodiments, the nutraceutical composition comprises a Bacterioides sp. e.g. Bacteroides fragilis and Bacteroides acidifaciens; a Clostridium sp. e.g. Clostridium innocuum; a Peptoclostridium sp. e.g. Clostridioides difficile; or a Fusobacterium sp. e.g., Fusobacterium mortiferum.

In certain embodiments, an emulsion or microemulsion may be used for the encapsulation of spirulina. Encapsulation in the food industry is a process in which one or more ingredients or additives (core) are coated with an edible capsule. The use of liposomes and microemulsions are among the many forms of encapsulation of food ingredients. Encapsulation consists of a sort of microscopic lipid vesicles, where due to the lipophilic and hydrophilic portion of its constituents, substances of various natures can be encapsulated, and the hydrophilic substances stay in the aqueous compartment and the lipophilic components are inserted or adsorbed on the membrane or surface of the hydrophilic substance. Sonication and homogenization processes may be used in the encapsulation of a protein source, such as the cyanobacterium Spirulina platensis, formed out by the thin-film hydration method. Liposome or micelle emulsions and microemulsions may be prepared using an appropriate emulsifier such as phosphatidylcholine or microemulsifier such as Plural® Oleique and sonicated at, e.g. 60° C. for e.g. 30 min. In certain embodiments, a liposome emulsion may be prepared by homogenizing a phosphatidylcholine, or a microemulsion may be prepared by homogenizing a polyglycerol ester of oleic acid, at e.g. 10,000 rpm for e.g. 15 min. The average size, encapsulation efficiency, and particle morphology is then determined. Either sonication or homogenization may be used to obtain nanometric size particles. In certain embodiments, an emulsion or microemulsion is formed with homogenization. In certain embodiments, an emulsion or microemulsion is formed with sonication.

In certain embodiments, a component in the nutraceutical composition is encapsulated. In certain embodiments, the spirulina is encapsulated. In certain embodiments, the spirulina is encapsulated with an emulsion or with a microemulsion. In certain embodiments, the emulsion or microemulsion comprises a polyglycerol ester of oleic acid. In certain embodiments, the microemulsion comprises glyceryl dibehenate. In certain embodiments, the microemulsion comprises Compritol 888 ATO. In certain embodiments, the microemulsion comprises di- and triglyceride esters of fatty acids. In certain embodiments, the microemulsion comprises Gelucire 43/01. In certain embodiments, the emulsion or microemulsion comprises a phosphatidyl choline. In certain embodiments the emulsion or microemulsion comprises a beta-glucan.

In certain embodiments, the probiotic is encapsulated. In certain embodiments, the probiotic is encapsulated with a polysaccharide.

Compositions—Specific

TABLE 1
Four fecal samples from three members of a Severe Acute Malnutritian (SAM) trial

| | Age of | | Time since | Anthropometry at time | | |
|---|---|---|---|---|---|---|
| | Donor | | discharge | of sample collection | | |
| Donor | (months) | Gender | from hospital | WHZ | WAZ | HAZ |
| 1 | 19.2 | M | 5 months | −2.73 | −2.71 | −1.59 |
| 2 | 19.8 | M | 6 months | −1.78 | −2.34 | −2.23 |
| 3 | 18.1 | M | 5 months | −1.9 | −2.18 | −1.71 |
| 4 | 13.3 | M | 1 week prior | −3.27 | −3.44 | −2.35 |

TABLE 2A
(T2A) Mirpur-18 (M18) base diet

| M18 base diet | Final weight (%) |
|---|---|
| Cooked rice | 41.8 |
| Whole milk powder | 13.9 |
| Cooked lentils | 16.2 |
| Cooked potato | 6.9 |
| Cooked spinach | 6.3 |
| Cooked Onion (yellow) | 3.7 |
| Soybean oil | 3.7 |
| Sweet pumpkin | 6 |
| Salt (iodized) | 0.5 |
| Turmeric | 0.5 |
| Garlic | 0.5 |
| Total | 100 |

TABLE 2B
(T2B) Supplemented M18 Diet

| Supplemented M18 | Final weight (%) |
|---|---|
| Spirulina | 5 |
| Flaxseed | 2.5 |
| Amaranth | 10 |
| Micronutrients: | 0.025 |
| Folic acid-0.8 μg | |
| Vitamin B3-30 μg | |
| Vitamin B6-2.5 μg | |
| Vitamin C-760 μg | |
| Vitamin D-0.16 μg | |
| Vitamin E-80 μg | |
| Zinc-40 μg | |
| Sub-total | 17.525 |
| (amaranth, flaxseed, spirulina, | |
| rnicronutrients mix) | |
| M18 Base | 82.475 |
| Total | 100 |

In certain embodiments, a base diet comprises variations, by weight, of the any of the M18 ingredients described in Table 2A. In certain embodiments, for example, cooked rice amounts may range from about 30% to about 45%, milk powder may range from about 8% to about 20%, cooked lentils may range from about 10% to about 20%, cooked potato may range from may range from about 5% to about 10%, cooked spinach may range from about 5% to about 10%, cooked onion may range from about 2% to about 5%, soybean oil may range from about 2% to about 5%, sweet pumpkin may range from about 4% to about 10%, salt may range from about 0.25% to about 0.7%, turmeric may range from about 0.2% to about 1%, and garlic may range from about 0.2% to about 2%.

In certain embodiments, a base diet comprises variations by component, of any of the M18 ingredients described in Table 2A. In certain embodiments, for example, cooked rice may substituted with cooked barley, cooked quinoa, cooked wheat bulger, cooked oatmeal, cooked wheat bran, cooked tapioca and combinations thereof; milk powder may be substituted with buttermilk powder, coconut milk powder, soy milk powder, rice milk powder, potato milk powder, protein powder or combinations thereof; cooked lentils may be substituted with cooked chickpeas, cooked beans, cooked peas, almond paste, peanut butter or combinations thereof; cooked potato may be substituted with cooked turnip, cooked beets, cooked carrots or combinations thereof; cooked spinach may be substituted with cooked mustard greens, cooked kale, cooked turnip greens, cooked dandelion greens, cooked bok choy, cooked cabbage, cooked brussel sprouts, cooked broccoli or combinations thereof; cooked onion may be substituted with cooked leeks, cooked scallions, cooked cabbage or combinations thereof; soybean oil may be substituted with corn oil, coconut oil, peanut oil, sesame oil, rapeseed oil, sunflower oil, olive oil, canola oil, safflower oil, grapeseed oil, or combinations thereof; sweet pumpkin may be substituted with yams, various squash, carrots, beets or combinations thereof; salt may be iodized NaCl, rock salt such as Himalayan salt, sea salt or combinations thereof, turmeric may be substituted with ground ginger, curry powder, cumin, mustard powder, or combinations thereof; and garlic may be substituted with leeks, spring onions, scallions or combinations thereof.

In certain embodiments, a base diet comprises variations, by weight, and variations by component, of any of the M18 ingredients described in Table 2A. In certain embodiments, for example, Cooked rice amounts may range from about 30% to about 45%, milk powder may range from about 8% to about 20%, cooked lentils may range from about 10% to about 20%, cooked potato may range from may range from about 5% to about 10%, cooked spinach may range from about 5% to about 10%, cooked onion may range from about 2% to about 5%, soybean oil may range from about 2% to about 5%, sweet pumpkin may range from about 4% to about 10%, salt may range from about 0.25% to about 0.7%, turmeric may range from about 0.2% to about 1%, and garlic may range from about 0.2% to about 2%.

In certain embodiments, there is provided a nutraceutical composition comprising

### Probiotic (−) Nutraceutical Composition I (or Simply Nutraceutical Composition I)

| Nutraceutical Composition I | Dry weight (%) |
|---|---|
| Spirulina | 5 |
| Flaxseed | 2.5 |
| Amaranth | 10 |
| Micronutrients | 0.025 |
| Probiotic | 0 |

plus a base diet to total 100% final weight, wherein the micronutrients component comprises i) vitamins B3, B6, C, D, (e.g. D3), E and B9 and ii) minerals comprising magnesium, selenium and zinc.

In certain embodiments, there is provided a nutraceutical composition comprising

### Probiotic (−) Nutraceutical Composition II (or Simply Nutraceutical Composition II)

| Nutraceutical Composition II | Dry weight (%) |
|---|---|
| Spirulina | 5-10 |
| Flaxseed | 1-4 |
| Amaranth | 5-15 |
| Micronutrients | 0.02-0.06 |
| Probiotic | 0 |

plus a base diet to total 100% final weight, wherein the micronutrients component comprises i) vitamins B3, B6, C, D (e.g. D3), E and B9 and ii) minerals comprising magnesium, selenium and zinc.

In certain embodiments, there is provided a nutraceutical composition comprising

### Probiotic (−) Nutraceutical Composition III (or Simply Nutraceutical Composition III)

| Nutraceutical Composition III | Dry weight (%) |
|---|---|
| Spirulina | 5-10 |
| Flaxseed | 3 |
| quinoa | 8 |
| Amaranth | 12 |
| Micronutrients | 0.05 |
| Probiotic | 0 |

plus a base diet to total 100% final weight, wherein the micronutrients component comprises i) vitamins B3, B6, C, D, (e.g. D3), E and B9 and ii) minerals comprising iron, magnesium, manganese, selenium and zinc.

In certain embodiments, there is provided a nutraceutical composition comprising

### Probiotic (−) Nutraceutical Composition IV (or Simply Nutraceutical Composition IV)

| Nutraceutical Composition IV | Dry weight (%) |
|---|---|
| Spirulina | 5 |
| Flaxseed | 3 |
| Amaranth | 10 |
| quinoa | 5-10 |
| Micronutrients | 0.05 |
| Probiotic | 0 |

plus a base diet to total 100% final weight, wherein the micronutrients component comprises i) optionally any of vitamins A, B3, B6, C, D (e.g. D3), E and B9 and ii) minerals comprising iron, magnesium, manganese, selenium and zinc.

In certain embodiments, there is provided a nutraceutical composition comprising

### Probiotic (−) Nutraceutical Composition V (or Simply Nutraceutical Composition V)

| Nutraceutical Composition V | Dry weight (%) |
|---|---|
| Spirulina | 5-10 |
| Flaxseed | 2-5 |
| Amaranth | 5-15 |
| quinoa | 5-15 |
| oat | 5-15 |
| Micronutrients | 0.05 |
| Probiotic | 0 |

plus a base diet to total 100% final weight, wherein the micronutrients component comprises i) vitamins A, B3, B6, C, D (e.g. D3), E and B9 and ii) minerals comprising iron, magnesium, manganese, selenium and zinc.

In certain embodiments, there is provided a nutraceutical composition comprising

### Probiotic (+) Nutraceutical Composition VI (or Simply Nutraceutical Composition VI)

| Nutraceutical Composition VI | Dry weight (%) |
|---|---|
| Spirulina | 5 |
| Flaxseed | 2.5 |
| Amaranth | 10 |
| Micronutrients | 0.025 |
| Probiotic | 0.02-5 |

plus a base diet to total 100% final weight, wherein the micronutrients component comprises i) vitamins B3, B6, C, D (e.g. D3), E and B9 and ii) minerals comprising magnesium, selenium and zinc; and the probiotic comprises a Lactobacillus sp. e.g. Lactobacillus rhamnosus, a Bacterioides sp., e.g. Bacteroides fragilis or Bacteroides acidifaciens; a non-toxigenic Clostridium sp., e.g. a non-toxigenic Clostridium innocuum sp. or a non-toxigenic Clostridioides difficile sp.; a Fusobacterium sp., e.g., Fusobacterium varium or Fusobacterium mortiferum, or a combination thereof. More particularly, the non-toxigenic Clostridioides difficile species does not possess genes encoding glucosylating exotoxins TcdA and TcdB.

In certain embodiments, there is provided a nutraceutical composition comprising

### Probiotic (+) Nutraceutical Composition VII (or Simply Nutraceutical Composition VII)

| Nutraceutical Composition VII | Dry weight (%) |
|---|---|
| Spirulina | 5-10 |
| Flaxseed | 1-5 |
| Amaranth | 5-15 |
| Micronutrients | 0.02-0.06 |
| Probiotic | 0.02-5 |

plus a base diet to total 100% final weight, wherein the micronutrients component comprises i) vitamins B3, B6, C, D (e.g. D3), E and B9 and ii) minerals comprising magnesium, selenium and zinc; and wherein the probiotic comprises a Lactobacillus sp. a Bacillus sp., a Bifidobacterium sp., an Enterococcus sp., a Lactococcus sp., a Pediococcus sp., a Saccharomyces sp., a Streptococcus sp., a Bacteroides sp., a non-toxigenic Clostridium sp., a Peptoclostridium sp., a Fusobacterium sp., or a combination thereof. Particularly, the probiotic comprises a Bacteroides sp., a Parabacteroides sp., a non-toxigenic Clostridium sp., a Peptoclostridium sp., a Fusobacterium sp. or a combination thereof. More particularly, the probiotic comprises a Bacteroides acidifaciens species, a Bacteroides fragilis species, a non-toxigenic Clostridioides difficile species, a non-toxigenic Clostridium innocuum species, a Fusobacterium mortiferum species or a combination of various strains thereof. More particularly, the non-toxigenic Clostridioides difficile species does not possess genes encoding glucosylating exotoxins TcdA and TcdB.

In certain embodiments, there is provided a nutraceutical composition comprising

### Probiotic (+) Nutraceutical Composition VIII (or Simply Nutraceutical Composition VIII)

| Nutraceutical Composition VIII | Dry weight (%) |
|---|---|
| Spirulina | 5-10 |
| Flaxseed | 1-5 |
| quinoa | 5-15 |
| Amaranth | 5-15 |
| Micronutrients | 0.02-0.06 |
| Probiotic | 0.02-5 |

plus a base diet to total 100% final weight, wherein the micronutrients component comprises i) vitamins B3, B6, C, D (e.g. D3), E and optionally B9 and ii) minerals comprising iron, magnesium, manganese, selenium and zinc; and wherein the probiotic comprises a Lactobacillus sp. a Bacillus sp., a Bifidobacterium sp., an Enterococcus sp., a Lactococcus sp., a Pediococcus sp., a Saccharomyces sp., a Streptococcus sp., a Bacteroides sp., a non-toxigenic Clostridium sp., a Peptoclostridium sp., a Fusobacterium sp., or a combination thereof. Particularly, the probiotic comprises a Bacteroides sp., a Parabacteroides sp., a non-toxigenic Clostridium sp., a Peptoclostridium sp., a Fusobacterium sp. or a combination thereof. More particularly, the probiotic comprises a Bacteroides acidifaciens species, a Bacteroides fragilis species, a non-toxigenic Clostridioides difficile species, a non-toxigenic Clostridium innocuum species, a Fusobacterium mortiferum species or a combination of various strains thereof. More particularly, the non-toxigenic Clostridioides difficile species does not possess genes encoding glucosylating exotoxins TcdA and TcdB.

In certain embodiments, there is provided a nutraceutical composition comprising

### Probiotic (+) Nutraceutical Composition IX (or Simply Nutraceutical Composition IX)

| Nutraceutical Composition IX | Dry weight (%) |
|---|---|
| Spirulina | 5-10 |
| Flaxseed | 1-5 |
| Amaranth | 5-15 |
| quinoa | 5-15 |
| Micronutrients | 0.02-0.05 |
| Probiotic | 0.02-4 |

plus a base diet to total 100% final weight, wherein the micronutrients component comprises i) vitamins A, B3, B6, C, D (e.g. D3), E and B9 and ii) minerals comprising iron, magnesium, manganese, selenium and zinc and the probiotic comprises a Lactobacillus sp. a Bacillus sp., a Bifidobacterium sp., an Enterococcus sp., a Lactococcus sp., a Pediococcus sp., a Saccharomyces sp., a Streptococcus sp., a Bacteroides sp., a non-toxigenic Clostridium sp., a Peptoclostridium sp., a Fusobacterium sp., or a combination thereof. Particularly, the probiotic comprises a Bacteroides sp., a Parabacteroides sp., a non-toxigenic Clostridium sp., a Peptoclostridium sp., a Fusobacterium sp. or a combination thereof. More particularly, the probiotic comprises a Bacteroides acidifaciens species, a Bacteroides fragilis species, a non-toxigenic Clostridioides difficile species, a non-toxigenic Clostridium innocuum species, a Fusobacterium mortiferum species or a combination of various strains thereof. More particularly, the non-toxigenic Clostridioides difficile species does not possess genes encoding glucosylating exotoxins TcdA and TcdB.

In certain embodiments, there is provided a nutraceutical composition comprising

### Probiotic (+) Nutraceutical Composition X (or Simply Nutraceutical Composition X)

| Nutraceutical Composition X | Dry weight (%) |
|---|---|
| Spirulina | 5-10 |
| Flaxseed | 2-5 |
| Amaranth | 5-15 |
| quinoa | 5-15 |
| Oat | 5-15 |
| Micronutrients | 0.02-0.05 |
| Probiotic | 0.02-4 |

plus M18 base diet to total 100% final weight, wherein the micronutrients component comprises i) vitamins A, B3, B6, C, D (e.g. D3), E and B9 and ii) minerals comprising iron, magnesium, manganese, selenium and zinc and the probiotic comprises a Lactobacillus sp. a Bacillus sp., a Bifidobacterium sp., an Enterococcus sp., a Lactococcus sp., a Pediococcus sp., a Saccharomyces sp., a Streptococcus sp., a Bacteroides sp., a non-toxigenic Clostridium sp., a Peptoclostridium sp., a Fusobacterium sp., or a combination thereof. Particularly, the probiotic comprises a Bacteroides sp., a Parabacteroides sp., a non-toxigenic Clostridium sp., a Peptoclostridium sp., a Fusobacterium sp. or a combination thereof. More particularly, the probiotic comprises a Bacteroides acidifaciens species, a Bacteroides fragilis species, a non-toxigenic Clostridioides difficile species, a non-toxigenic Clostridium innocuum species, a Fusobacterium mortiferum species or a combination of various strains thereof. More particularly, the non-toxigenic Clostridioides difficile species does not possess genes encoding glucosylating exotoxins TcdA and TcdB.

In certain embodiments, the nutraceutical compositions of the invention described herein comprise any of the Probiotic (+) Nutraceutical Compositions I, II, III, IV, V, VI, VII, VIII, IX or X. Certain embodiments of the invention comprise a nutraceutical composition comprising any of the Probiotic (+) Nutraceutical Compositions I, II, III, IV, V, VI, VII, VIII, IX or X, wherein the nutraceutical composition comprises a microbiota-associated metabolite that capable of modulating an enhanced CT specific-IgA response in a mammal. In certain embodiments, the microbiota-associated metabolite that is capable of modulating an enhanced CT-specific IgA response in a mammal is tryptophyl-histidine. In certain embodiments, the microbiota-associated metabolite that is capable of modulating an enhanced IgA response in a mammal is proline betaine. In certain embodiments, the microbiota-associated metabolite that is capable of modulating an enhanced IgA response in a mammal are metabolites with masses and corresponding retention times (in min) as shown in FIG. 13C.

In certain embodiments, the microbiota-associated metabolite that is capable of modulating an enhanced IgA response in a mammal is a metabolite selected from:

a metabolite having a mass of 213.0995 and a retention time of 5.81;

a metabolite having a mass of 604.2226 and a retention time of 10.12;

a metabolite having a mass of 707.3953 and a retention time of 10.06;

a metabolite having a mass of 197.1045 and a retention time of 6.74;

a metabolite having a mass of 341.148 and a retention time of 4.04;

a metabolite having a mass of 350.1541 and a retention time of 1.37;

a metabolite having a mass of 731.4002 and a retention time of 10.22;

a metabolite having a mass of 385.218 and a retention time of 4.65;

a metabolite having a mass of 268.204 and a retention time of 12.35;

a metabolite having a mass of 329.0942 and a retention time of 2.62;

a metabolite having a mass of 366.255 and a retention time of 14.52;

a metabolite having a mass of 315.8375 and a retention time of 8.62;

a metabolite having a mass of 326.1264 and a retention time of 4.97;

a metabolite having a mass of 320.1737 and a retention time of 7.51;

a metabolite having a mass of 707.3004 and a retention time of 6.84;

a metabolite having a mass of 284.142 and a retention time of 7.67;

a metabolite having a mass of 336.1699 and a retention time of 6.58;

a metabolite having a mass of 266.1883 and a retention time of 11.63;

a metabolite having a mass of 181.0725 and a retention time of 6.58;

a metabolite having a mass of 306.1453 and a retention time of 5.98;

a metabolite having a mass of 345.7956 and a retention time of 8.62;

a metabolite having a mass of 336.169 and a retention time of 6.92;

a metabolite having a mass of 134.0945 and a retention time of 4.14;

a metabolite having a mass of 214.094 and a retention time of 2.92;

a metabolite having a mass of 309.1076 and a retention time of 3.52;

a metabolite having a mass of 309.1076 and a retention time of 3.53;

a metabolite having a mass of 336.1693 and a retention time of 6.58;

a metabolite having a mass of 284.199 and a retention time of 11.62;

a metabolite having a mass of 348.1723 and a retention time of 7.18;

a metabolite having a mass of 328.1505 and a retention time of 4.58;

a metabolite having a mass of 296.1983 and a retention time of 11.69;

a metabolite having a mass of 266.131 and a retention time of 7.67;

a metabolite having a mass of 300.1077 and a retention time of 4.78;

a metabolite having a mass of 302.162 and a retention time of 6.58;

a metabolite having a mass of 317.1659 and a retention time of 9.44;

a metabolite having a mass of 320.1737 and a retention time of 7.1;

a metabolite having a mass of 336.1678 and a retention time of 6.92;

a metabolite having a mass of 390.2 and a retention time of 3.83; or

a metabolite having a mass of 253.142 and a retention time of 3.82.

Nutraceutical Administration

In an embodiment, the nutraceutical compositions described herein may be fed or administered 1×-3× daily, or 1×-3× weekly, biweekly or monthly to a child during the first 1000 days of life, or as needed, including before, at the same time or after vaccination. In an embodiment, the nutraceutical compositions described herein may be fed or administered 1×-3× daily, or 1×-3× weekly, biweekly or monthly to a pregnant female throughout the pregnancy and also to a lactating female post-delivery. In an embodiment the nutraceutical compositions described herein may be fed or administered 1×-3× daily, or 1×-3× weekly, biweekly or monthly to an elderly person, as needed, including before or after vaccination. The frequency and amount of the dosage to be administered will be determined based on one or more factors including age, weight and health of the child, pregnant female, lactating female or elderly person receiving the nutraceutical composition. Similarly, the time of day for administration of the nutraceutical compositions described herein will be determined based on need, health, age and weight of the subject receiving the nutraceutical composition.

Nutraceutical compositions as described herein will generally be in powder form which may be reconstituted in a liquid, but may also be given as a gel, a paste, tablet, capsule, or liquid formulation, as appropriate.

### Examples—Nutraceutical Effect on Immune Response

### Example 1: Preclinical Experiments in Gnotobiotic Mice Designed to Identify Bacterial Mediators of the Probiotic (−) Nutraceutical Composition I Supplement-Associated Increase in Oral Vaccine Response

Study 1

Mice Fed Probiotic (−) Nutraceutical Composition I Followed by Immunization with CT/OVA

A gnotobiotic mouse model was used to test whether microbiota obtained from undernourished children are associated with impaired oral vaccine responses and to determine whether the probiotic (−) Nutraceutical composition I can produce improved immune responses to oral vaccination with Cholera Toxin (CT).

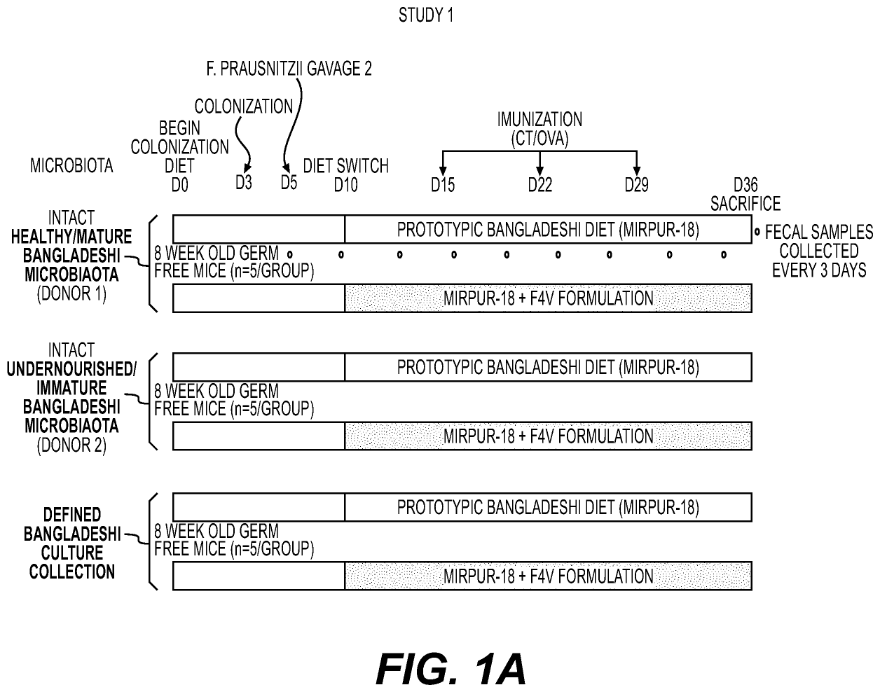

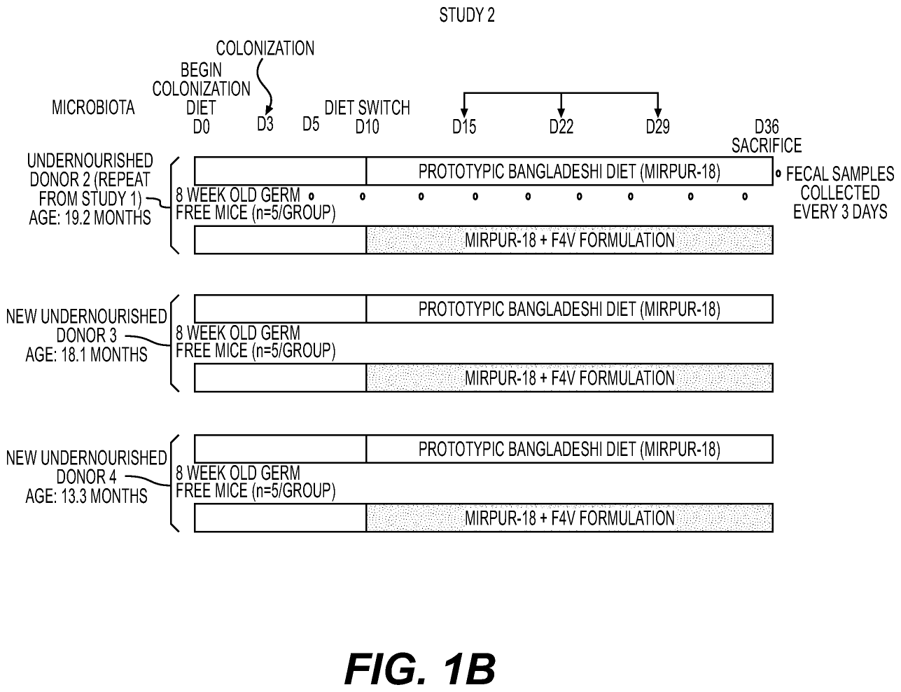

As a first preclinical experiment (study design shown in FIG. 1A), 8-week-old male germ-free C57BL/6J mice were fed a nutritionally deficient diet (Mirpur-18; M18) representative of that consumed by the human microbiota donor population for 3 days prior to colonization. Mice were then colonized by oral gavage with one of three different microbial communities (n=10 animals/recipient group): intact uncultured microbiota from 2 Bangladeshi children described in Table 1 (Donor 1 and Donor 2) and a defined consortium of bacterial strains (culture collection) including age-discriminatory strains plus several SAM-associated taxa cultured from children living in Bangladesh (Gehrig et al., 2019).

Seven days after receiving these communities, each group of animals was divided into two subgroups: one subgroup was monotonously fed the M18 base (unsupplemented) diet ad libitum, and the other was switched to the probiotic (−) Nutraceutical Composition I (5% spirulina, 2.5% flaxseed, 10% amaranth and 0.025% micronutrients, dry weight) on top of the M18 base diet (also ad libitum). The composition of the supplemented M18 diet provided 200 mg spirulina/mouse/day and 75% of the RDA of a number of vitamins and minerals for a 12-month-old child. Tables 2A and 2B describe the formulations of the (M18 base diet and probiotic (−) Nutraceutical Composition I-supplemented M18 diet, respectively, and their nutritional analysis after irradiation. Gas chromatography-mass spectrometric (GC-MS) analysis was performed on cecal contents harvested from control germ-free mice monotonously fed the two diets for 15 days, and demonstrated statistically significant increases in cecal levels of multiple essential and non-essential amino acids as well as mono- and disaccharides in mice consuming the supplemented M18 diet.

All mice in all treatment arms received three oral vaccinations with a mixture of cholera toxin (CT) and chicken ovalbumin (OVA); vaccinations occurred 7 days apart, beginning on experimental day 15. At sacrifice 33 days after initial gavage (and 7 days after the final vaccination), anti-CT IgA levels were assessed by ELISA of feces and serum.

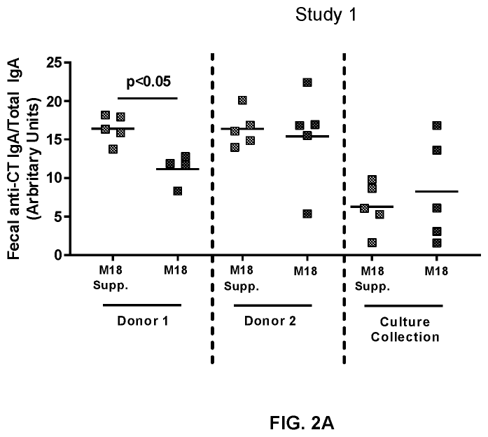

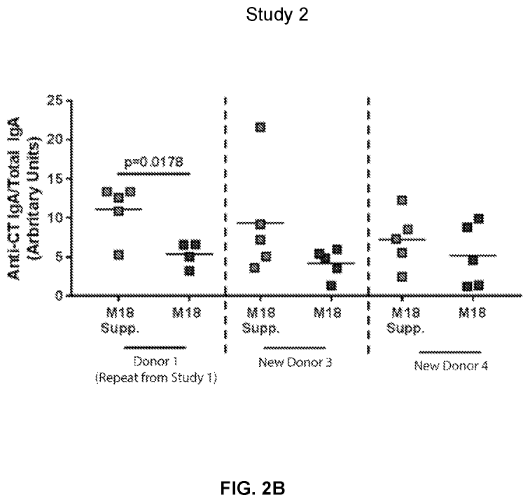

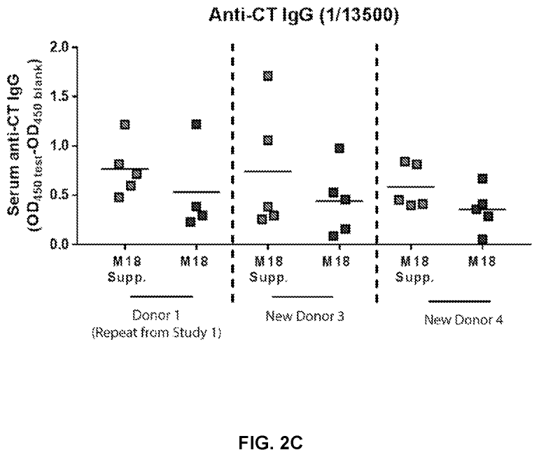

Probiotic (−) Nutraceutical Composition I supplementation of the M18 diet led to a significant increase in the anti-CT IgA response in the feces of gnotobiotic recipients of the microbiota from Donor 1, boosting the response to a level equivalent to that seen in recipients of microbiota from Donor 2. Supplementation did not produce a statistically significant effect on the anti-CT IgA response in the feces of recipients of the intact microbiota (Donor 2) or in recipients of the culture collection, despite clear differences in the magnitude of vaccine responsiveness between the two groups of animals (FIG. 2A).

No statistically significant differences were observed in serum anti-CT IgG responses between the 6 treatment groups suggesting that the beneficial effects of supplementation manifest locally in the intestine, rather than systemically (data not shown). 16S rDNA-based analysis of fecal samples collected at various times throughout the experiment revealed very minor changes in the relative abundances of bacterial community members within each given group of animals after the diet switch, indicating that the prebiotic supplement's enhancement of oral vaccination response to CT was not associated with a marked change in microbiota structure (data not shown).

Study 2

A follow-on study was performed of the same design to that of FIG. 1A, shown in FIG. 1B; one group of mice received the same “nutraceutical-responsive” microbiota sample from the donor that was employed in the first experiment (Donor 1). Other mice received two other microbiota from undernourished donors; Donor 3 and Donor 4 (Table 1 and FIG. 1B). Note that Donor 4 represents a microbiota sample from the Donor 2 used in Study 1 but obtained at a time point that was 6 months earlier (age: 13.3 mol) when the donor had exhibited significantly greater undernutrition; this earlier sample was referred to as Donor 4 in this second experiment and throughout, to avoid any confusion.

Assessment of fecal anti-CT IgA responses revealed that nutraceutical supplementation of the M18 diet in recipients of Donor 1 increased the CT-IgA vaccine response measured in feces, recapitulating the results obtained with the same sample in Study 1 (FIG. 2A). The recipients of the new Donor 3 who received the nutraceutical composition exhibited a slightly increased immune response compared with the mice that did not receive nutraceutical supplementation (compare left side squares to right side squares), although the difference was not statistically significant. However, no significant effects of the nutraceutical composition on vaccine response to CT was observed in recipients of the Donor 4, indicating that the increase of anti-CT IgA response was specific to Donor 1 rather than a general feature of all undernourished microbiota (FIG. 2B). As in Study 1, no significant diet-dependent differences in the anti-CT IgG response was observed in sera obtained from any of the recipient groups (FIG. 2C).

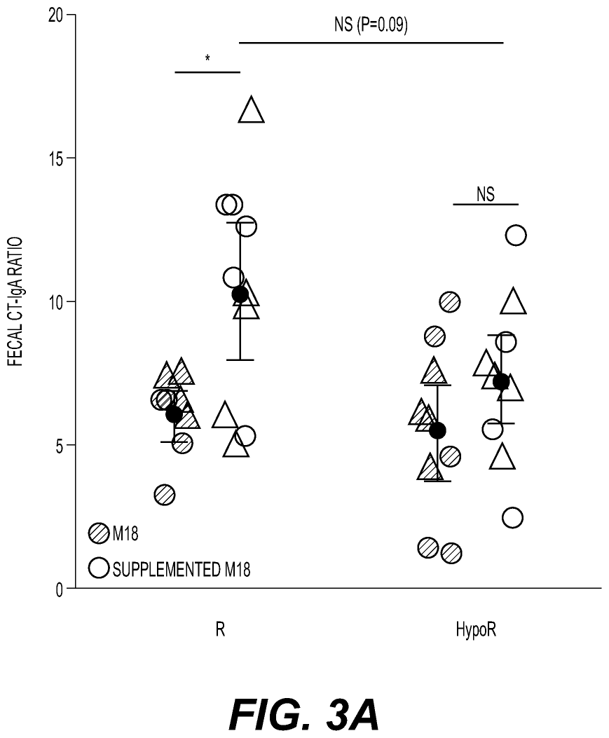

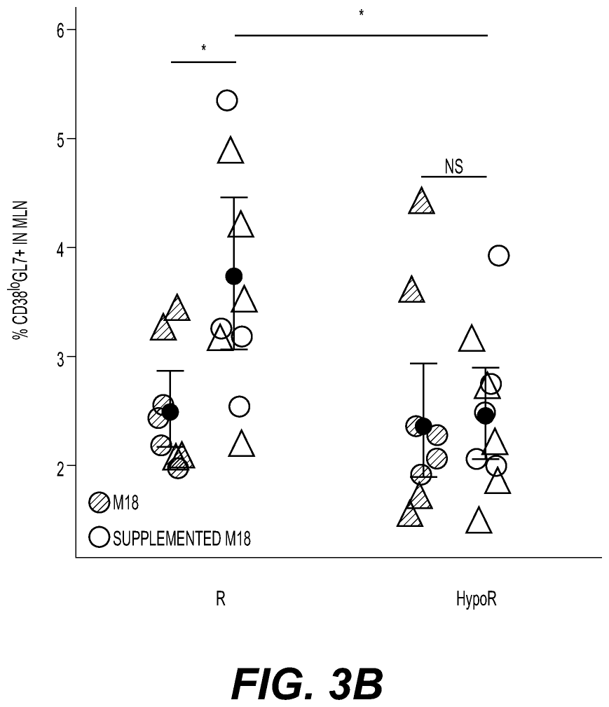

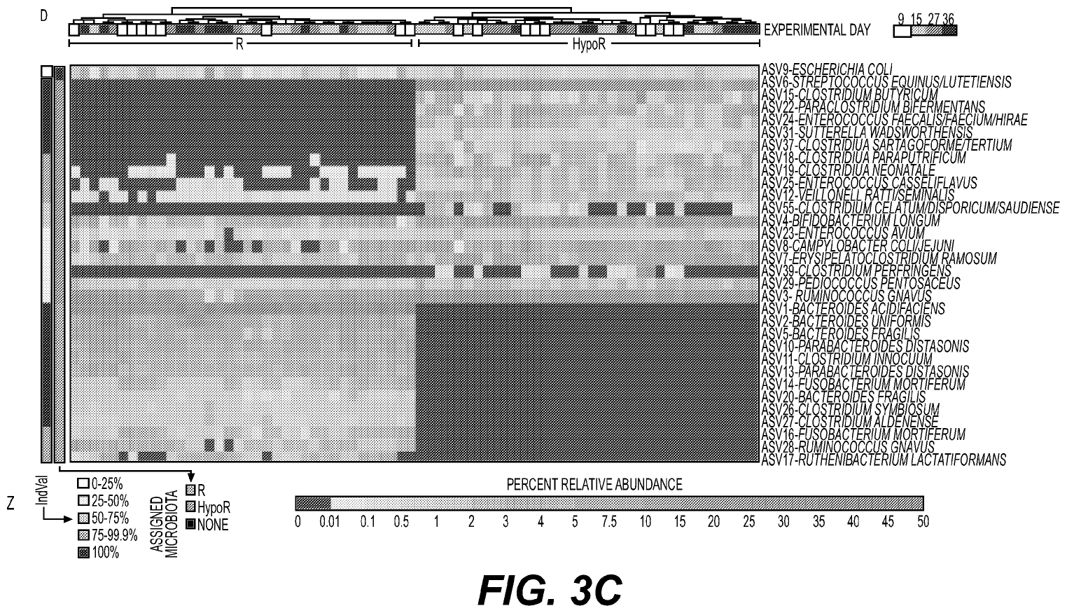

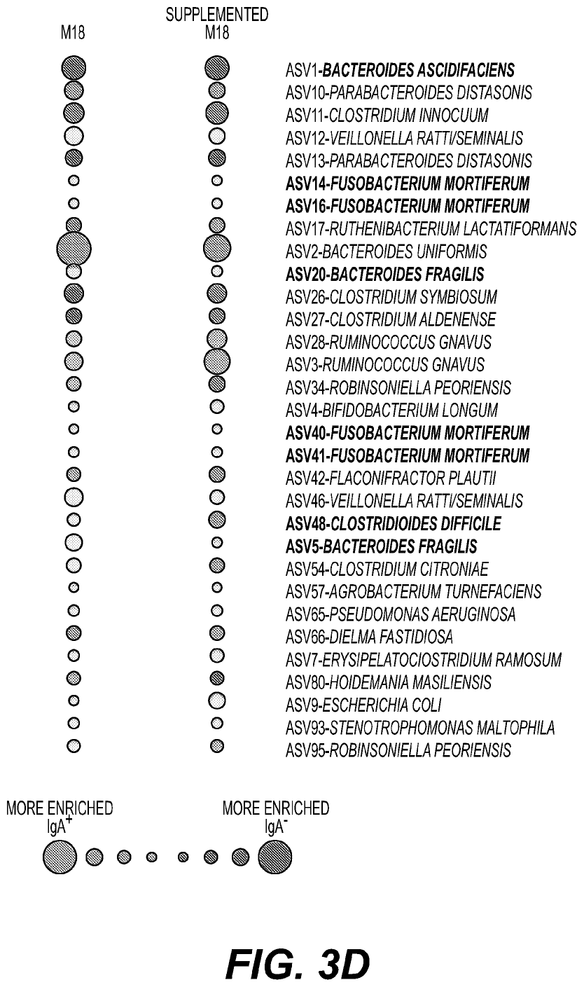

Analyzing responses in all mice colonized with microbiota from either Donor 1 and Donor 4 from both Study 1 and Study 2 confirmed that the anti-CT IgA response in the feces of the mice fed the supplemented M18 diet was higher than in mice fed unsupplemented M18 (linear mixed effects model; FIG. 3A). Among the four different post-SAM MAM microbiota tested, this difference only reached statistical significance in Donor 1-colonized mice (linear mixed-effects model followed by linear contrasts, P<0.05; note that total IgA levels in feces did not differ between treatment groups, either before or after vaccination). Mice colonized with this donor microbiota and fed supplemented M18 also had higher levels of germinal center B-cells (CD38^{lo} GL7⁺) in their mesenteric lymph nodes (MLN) relative to their unsupplemented M18-fed counterparts (FIG. 3B). In mice fed supplemented M18, The CT-IgA ratio and the percentage of CD38^{lo} GL7⁺ cells were higher in animals colonized with Donor 1 than in those colonized with Donor 4 (P=0.092 for CT-IgA ratio, P=0.011 for CD38^{lo} GL7⁺ cells) (FIG. 3A and FIG. 3B). There were no statistically significant effects on the representation of activated CD4⁺ T cells (CD44^{hi} CD62L^{lo}), Foxp3⁺ regulatory T cells (Tregs) or on levels of serum CT-specific IgG in Donor 1- and Donor 4-colonized mice. A similarly designed experiment involving germ-free mice showed no significant differences in the CT-IgA ratio or total IgA in feces between animals fed the M18 or supplemented M18 diets (n=5 mice/group) [CT-IgA ratio; 9.2±2.4 (mean±SEM) (M18) versus 9.2±3.4 (supplemented M18); total IgA, 1.3±0.4 (M18) versus 3.2±0.9 (supplemented M18); P=0.989, P=0.092, respectively (ANOVA)]. Together, these data indicate that immune responses to CT are influenced by both community composition and dietary context. Based on these observations, the Donor 1 and Donor 4 microbiota were designated supplement-responsive (R) and supplement-hypo-responsive (HypoR), respectively.

Using fecal samples obtained on experimental days 9, 15, 27 and 36 from animals fed the two diets, we performed indicator species analysis to identify bacterial taxa (amplicon sequence variants; ASVs) whose frequencies of detection and relative abundances differed between the R and HypoR communities. ISA assigns a strength of association (indicator species value) to each ASV in each community type (R or HypoR), calculated as the product of an ASV's frequency of detection, and mean relative abundance in a given community type, normalized to the sum of its mean abundances in both communities. This value ranges from zero, indicating that an ASV is never detected in a group, to 100%, indicating that it is always and only detected in a single group. Statistical significance was determined by permutation. ISA identified 30 ASVs that were significantly associated with R microbiota-colonized mice and 27 ASVs associated with mice that received the HypoR community. Among the taxa exclusively found in the transplanted R community were several members of Bacteroidales (Bacteroides fragilis, Bacteroides acidifaciens, Parabacteroides distasonis), three ASVs assigned to Fusobacterium mortiferum, and another ASV assigned to Clostridium innocuum. Streptococcus lutetiensis, Enterococcus and two members of the genus Clostridium were among those ASVs restricted to mice harboring the HypoR microbiota (FIG. 3C).

Permutational multivariate analysis of variance (PERMANOVA) was also applied to V4-16S rDNA datasets across the two experiments depicted in FIG. 1A and FIG. 1B with both the R (Donor 1) and HypoR (Donor 4) communities. Diet explained 5.3% and 28.9% of the variance (Bray-Curtis dissimilarities) between R and HypoR communities sampled at the end of the experiments, respectively (P=0.033 and P=0.0002). For the HypoR community, diet divided the samples along the first axis in a principal coordinates analysis. This axis was correlated with the relative abundances of 17 ASVs, 14 of which were significant indicator ASVs. The 13 HypoR-associated ASVs all had mean relative abundances that were either higher in HypoR- compared to R-colonized mice in both diet contexts, or were never detected in the R microbiota. The effect of supplementation on the bacterial composition of the HypoR community was largely attributable to increases in the relative abundance of ASV6 (S. lutetiensis; 18.2% versus 7.2% on M18) and to decreases in the relative abundances of ASV3 (R. gnavus; 17.8% versus 22.4%), ASV4 (Bifidobacterium; 11.3% versus 14.9%), and ASV7 (Erysipelatoclostridium ramosum; 6.6% versus 9.6%).

To examine whether M18 supplementation influenced the bacterial taxa that were targeted by gut mucosal IgAs, we performed BugFACS on fecal samples collected on experimental day 36 from R community-colonized mice. This method uses fluorescence-activated cell sorting to separate bacterial cells based on whether they are bound by host IgA (IgA+) or not (IgA−). Targeting of bacteria by IgA was similar in mice fed the two diets, with enrichment in the IgA+ fraction most significant for Bacteroides uniformis (ASV2) and Ruminococcus gnavus (ASVs 3 and 28); (FIG. 3D). B. acidifaciens (ASV1), P. distasonis (ASVs 9 and 13), Clostridium innocuum (ASV11), and Lachnospiraceae (ASV 24) were highly enriched in the IgA− fraction on both diets. PERMANOVA of Bray-Curtis dissimilarities showed that the different fractions (IgA+vs. IgA−) explained 45.2% of the total variance (P<0.001), while the diets and their interaction with the IgA+ and IgA− fractions were not significant (P>0.05) and collectively explained only 3.9% of the variation. Together, these results led us to conclude that the observed enhancement of CT-IgA response was not accompanied by detectable alterations in mucosal IgA targeting of bacterial members of the transplanted R community.

### Example 2: Effects of Co-Housing Mice Initially Colonized with the R or HypoR Microbiota

Study 3

Invasion of the HypoR Microbiota by Members of the R Community is Associated with Increased IgA Response to Oral Vaccination with CT

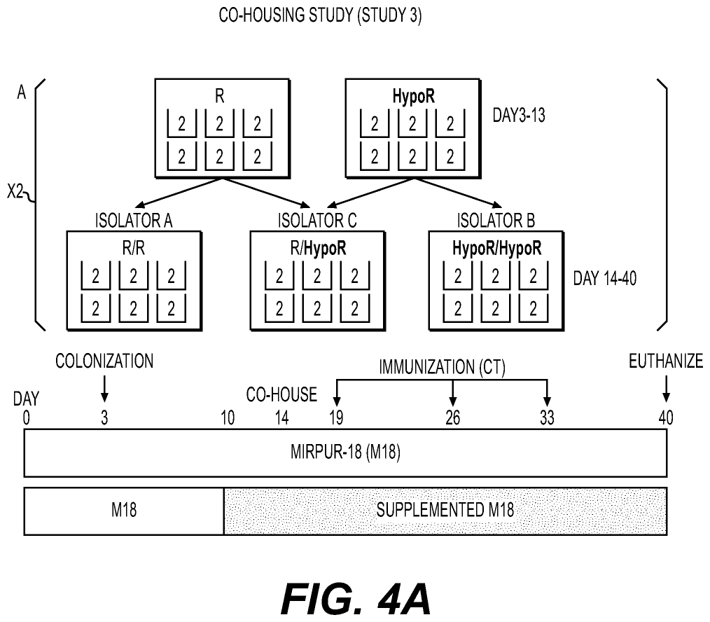

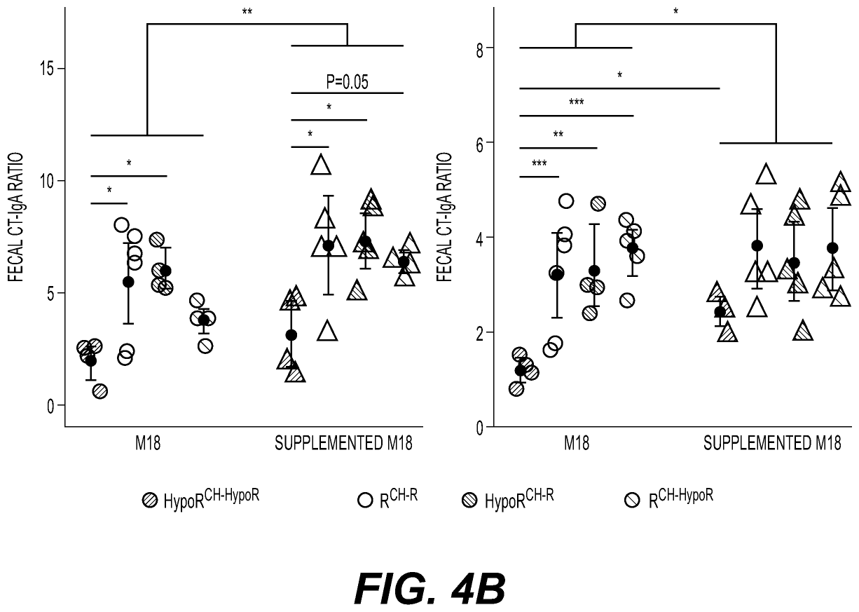

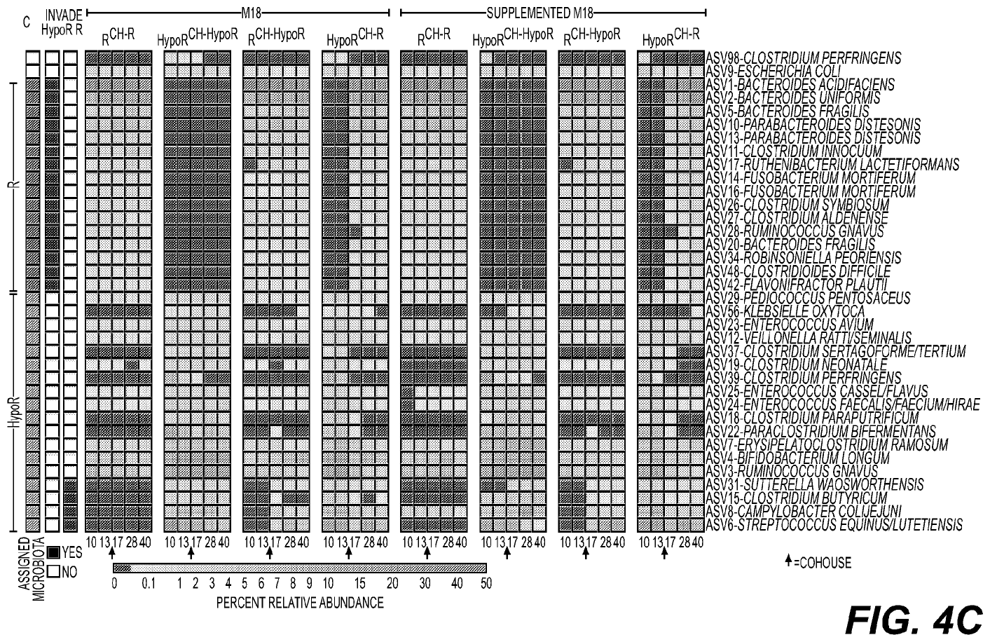

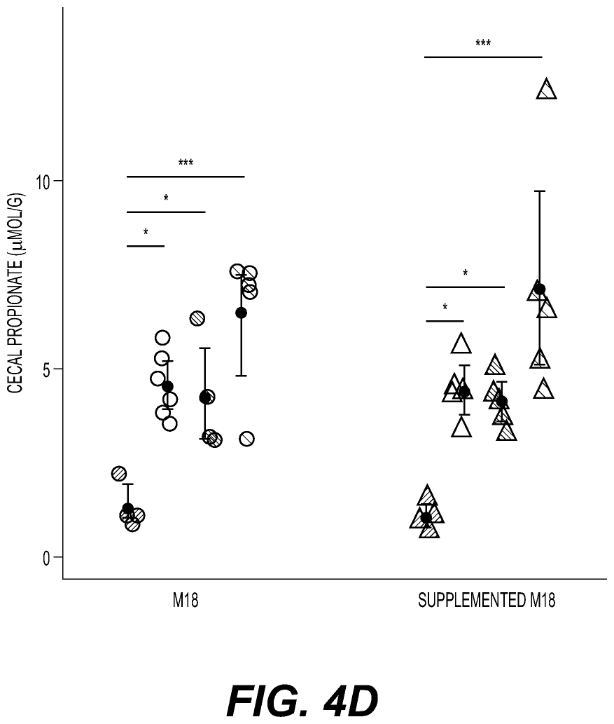

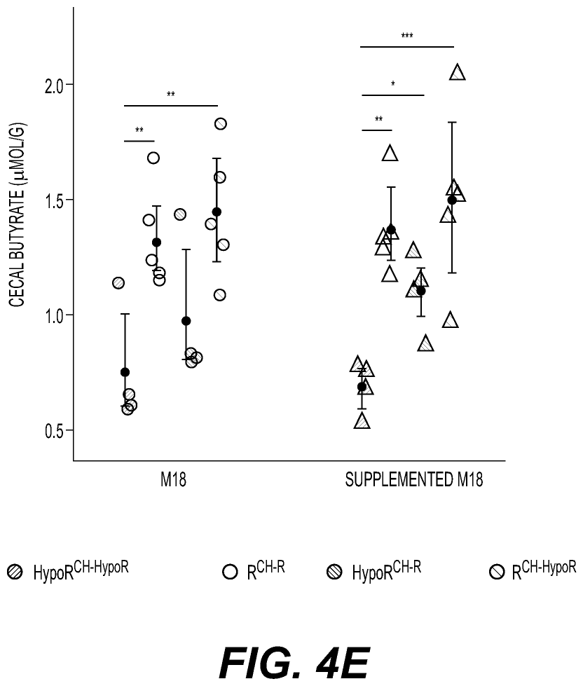

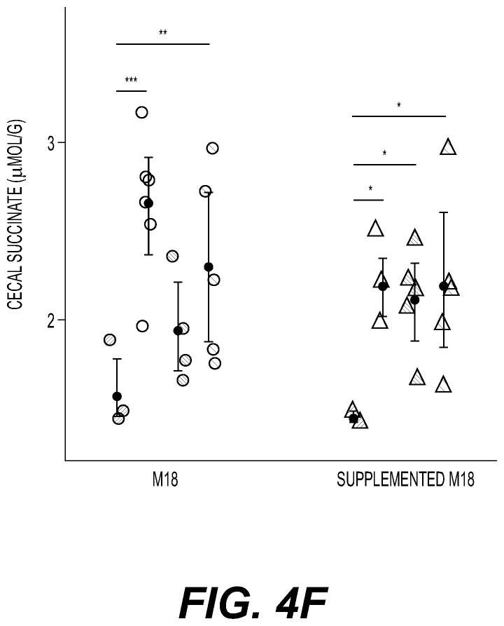

Co-housing Study 3 was performed to test the hypothesis that specific microbial taxa present in the R microbiota could invade and establish themselves in the transplanted HypoR community, leading to acquisition of a supplement-enhanced IgA response to oral CT vaccination. The experimental design for Study 3 is illustrated in FIG. 4A. Forty-eight 8-week-old, germ-free, male C57BL/6J mice consuming the unsupplemented M18 diet were colonized with either the R or HypoR communities (n=24 animals/microbiota). One week after colonization, 12 mice with each gut microbiota were switched to monotonous feeding with supplemented M18, while the other 12 mice from each group were maintained on unsupplemented M18. Four days later, half of each group were placed in a new isolator, where they were co-housed in pairs with a cagemate harboring either the same microbiota or the microbiota of the other donor, but maintained on the same diet. This resulted in four groups of mice with different microbial exposures: R-colonized mice cohoused with R-colonized mice (abbreviated R^{Ch-R}), R-colonized mice cohoused with HypoR-colonized mice (R^{Ch-HypoR}), HypoR-colonized mice cohoused with HypoR-colonized mice (HypoR^{Ch-HypoR}), and HypoR-colonized mice cohoused with R-colonized mice (HypoR^{Ch-R}). Mice then received three oral CT vaccinations at weekly intervals beginning five days after co-housing.

Consistent with earlier results, (i) mean fecal CT-IgA ratios were increased in mice colonized with the R microbiota relative to those harboring the HypoR community (linear model, linear contrasts of marginal means, P<0.001) and (ii) consuming supplemented M18 increased the CT-IgA ratio relative to M18 alone (linear model, P=0.005; FIG. 4B). Moreover, HypoR^{Ch-HypoR} mice consuming the supplemented M18 diet had lower fecal CT-IgA ratios when compared to mice that were first exposed to the R community (R^{Ch-R}; R^{Ch-HypoR}) or later exposed to it (HypoR^{Ch-R}). The percentages of activated CD4⁺ T cells (CD44^{hi} CD62L^{lo}), indicative of immune activation, and Tregs (Foxp3⁺) which are mediators of oral tolerance in MLN, did not differ between any of the eight treatment groups. Together, these findings led us to conclude that mucosal IgA responses to CT following oral vaccination can be enhanced by supplementation of the M18 diet and by the presence of specific members of the R community.

The frequency of germinal center CD38^{lo} GL7⁺ B cells in MLN was significantly higher in R^{Ch-R}, HypoR^{Ch-R} and R^{Ch-HypoR} mice compared to HypoR^{Ch-HypoR} controls while animals consumed the unsupplemented M18 diet. The HypoR group also manifested increases in these cells from diet supplementation alone without exposure to R community members (FIG. 4B). Consistent with these findings, the percentage of MLN CD38^{hi} IgD⁺ cells, which cannot produce IgA, exhibited the opposite pattern to that observed for MLN CD38^{lo} GL7⁺ cells. We concluded that under these experimental conditions, members of the R microbiota had a larger effect size on the germinal center memory B cell response than did the supplement.

V4-16S rDNA datasets were also generated from fecal samples collected from mice in all groups at various time points to determine the effects of co-housing on community composition (FIG. 4C). Invasion of an ASV in the R→HypoR direction was defined as follows: (i) the ASV increased from a relative abundance <0.1% before co-housing to a relative abundance ≥0.1% after co-housing in at least 75% of the HypoR^{Ch-R} mice; (ii) the ASV had a relative abundance ≥0.1% in at least one sample obtained from 75% of the mice in both the R^{Ch-R} and R^{Ch-HypoR} groups after co-housing, and (iii) the ASV was not detected at a relative abundance ≥0.1% in more than one HypoR^{Ch-HypoR} mouse after co-housing. For ASVs invading in the HypoR→R direction, the rules were modified to reflect gain by R^{Ch-HypoR} mice, and persistence in HypoR^{Ch-HypoR} and HypoR^{Ch-R} animals, and absence from R^{Ch-R} mice. Applying these criteria in each diet context independently (ASVs were considered as successful invaders if they met the criteria in either diet context), 23 ASVs invaded in the R→HypoR direction, including ASVs assigned to B. acidifaciens, B. uniformis, B. fragilis, Clostridium innocuum, Fusobacterium mortiferum, and Clostridioides difficile (FIG. 4C). Conversely, four ASVs invaded in the HypoR→R direction; these were assigned to S. lutetiensis, Campylobacter, C. butyricum, and Sutterella. Together, these findings suggest that the R microbiota contains strains able to augment the gut mucosal CT-IgA response in the context of the Nutraceutical Composition I-supplemented diet. Moreover, this enhanced immunogenicity can be transmitted to mice harboring a HypoR microbial community after invasion of their microbiota by taxa from R mice.

Acetate, propionate and butyrate are major products of colonic bacterial fermentation of dietary fibers that signal through G protein-coupled receptors (GPR41, GPR43 and GPR109a). A recent study demonstrated that acetate and butyrate can promote B cell differentiation and boost IgA responses to oral CT vaccination (Yang et al., (2019). Therefore, we used GC-MS to quantify short-chain fatty acids in cecal samples collected from each mouse in each group at the end of the co-housing experiment. Propionate, butyrate and succinate were all significantly higher in mice consuming the supplemented M18 diet that had been exposed to the R microbiota (either initially by gavage (R^{Ch-R}), or after co-housing (HypoR^{Ch-R})) compared to the HypoR^{Ch-HypoR} animals that had not been exposed (FIG. 4D-FIG. 4F). In contrast to microbial exposure, diet (unsupplemented versus supplemented) had no statistically significant effect on cecal levels of short chain fatty acids (FIG. 4D-FIG. 4F).

### Example 3: Invasion of the HypoR Microbiota by Cultured Members of the R Community Increases Fecal CT-IgA Ratios in Prebiotic-Supplemented Gnotobiotic Mice

Study 4

Testing the Effects of a Cultured Consortium of R-Derived R->HypoR Invaders on Vaccine Responses

To determine whether a defined consortium composed of cultured R->Hypo invaders from the R community could produce effects comparable to those observed in the co-housing experiments, we cultured and sequenced 5 bacterial strains (Bacteroides fragilis, Bacteroides acidifaciens, Clostridium innocuum, Clostridioides difficile, Fusobacterium mortiferum; ‘R culture consortium or 5memRCC’) from the R microbiota that were robust colonizers of gnotobiotic mice in both diet contexts and identified as R->HypoR invaders in the co-housing study (Study 3). The genomes of these 5 strains were sequenced and in silico metabolic reconstructions were performed. A search of the ‘virulence factors of pathogenic bacteria database’ revealed that the recovered strain of C. difficile does not possess the genes encoding the two principal glucosylating exotoxins, TcdA and TcdB, that are primarily responsible for mediating its pathogenic effects.

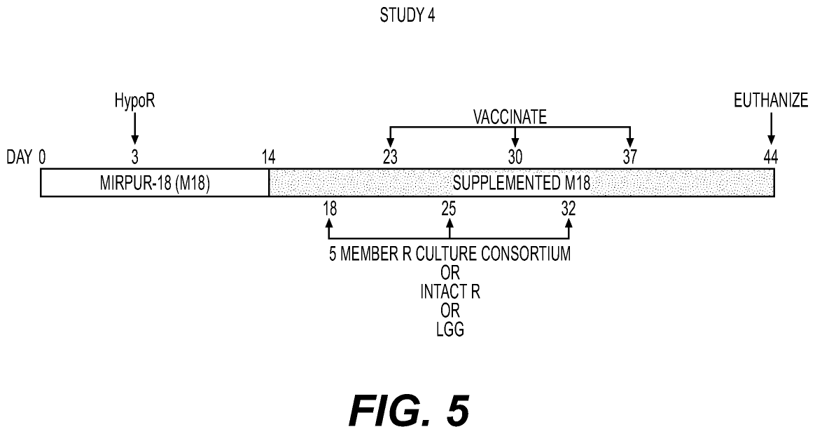

Study 4 was performed on 8-week-old C57BL/6J germ-free mice which were started on the unsupplemented M18 diet and colonized with the Donor 4 HypoR microbiota three days later (see FIG. 5). On experimental day 14, all mice were switched to the nutraceutical-supplemented M18 diet and then maintained on this diet for the duration of the experiment. On experimental day 18, mice were allocated into 1 of 4 arms (n=8 mice/arm) in different gnotobiotic isolators: (i) a control arm in which mice received no probiotic gavage (control HypoR microbiota group); (ii) an arm in which mice were gavaged with the intact R microbiota (+intact R microbiota group), (iii) an arm that in which mice were gavaged with the 5-member consortium of cultured bacterial strains from the R community (R culture consortium group/5memRCC), and (iv) an arm in which mice were gavaged with the Lactobacillus rhamnosus GG strain (LGG; 1×10⁹ CFU) (+L. rhamnosus GG (LGG) group). The CT vaccination regimen used in this experiment was similar to that used in previous studies with the exception that 5 days before each vaccination, animals in the experimental arms (ii-iv) were re-gavaged with the respective probiotic community (for a total 3 separate probiotic doses).

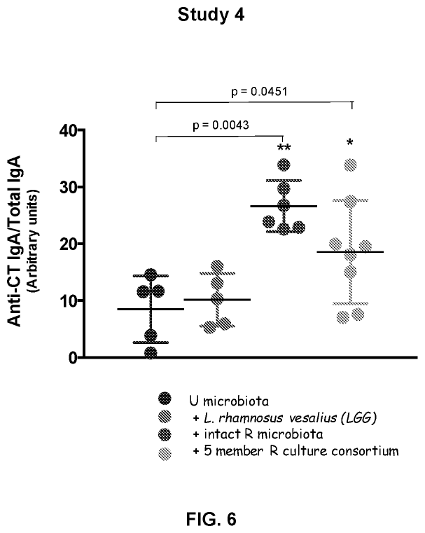

Assessment of fecal anti-CT IgA responses by ELISA revealed that as expected, mice initially colonized with the HypoR microbiota that subsequently received the intact uncultured R microbiota exhibited a statistically significant enhancement of their response to vaccination compared to the control group that were colonized with the HypoR community alone (FIG. 6). Importantly, mice that received the 5-member probiotic consortium comprised of R strains identified as R->HypoR invaders in the co-housing study, also exhibited a significant augmentation in vaccine response. In contrast, mice that received L. rhamnosus GG (LGG) did not show an increased vaccine response over that produced in mice colonized with the HypoR microbiota alone.

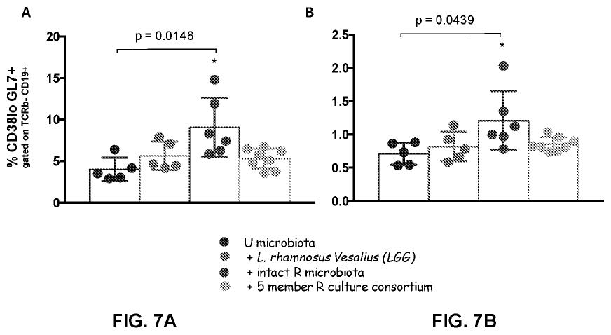

Using FACS of immune cells prepared from tissues harvested at sacrifice, an increase in the activated/memory B cell population was observed in both the mesenteric lymph nodes (FIG. 7A) and in the spleens (FIG. 7B) of mice that received the intact R microbiota. Interestingly, the same increase was not observed in recipients of the cultured R consortium. However, given that these cells were sorted using markers present on all activated/memory B cells (CD38^{lo} GL7⁺, previously gated on CD19⁺ TCRbeta⁻), it is not possible to exclude the possibility that there is an increase of memory B cells specific for the cholera toxin antigen that are not distinguishable using this approach. No differences in systemic vaccine responses, or in Treg cells in the mesenteric lymph node were observed in any of the experimental groups (data not shown).

Increased Fecal CT-IgA Ratios in Nutraceutical Composition I-Supplemented Gnotobiotic Mice was Associated with Invasion of the HypoR Microbiota by Members of the 5memRCC

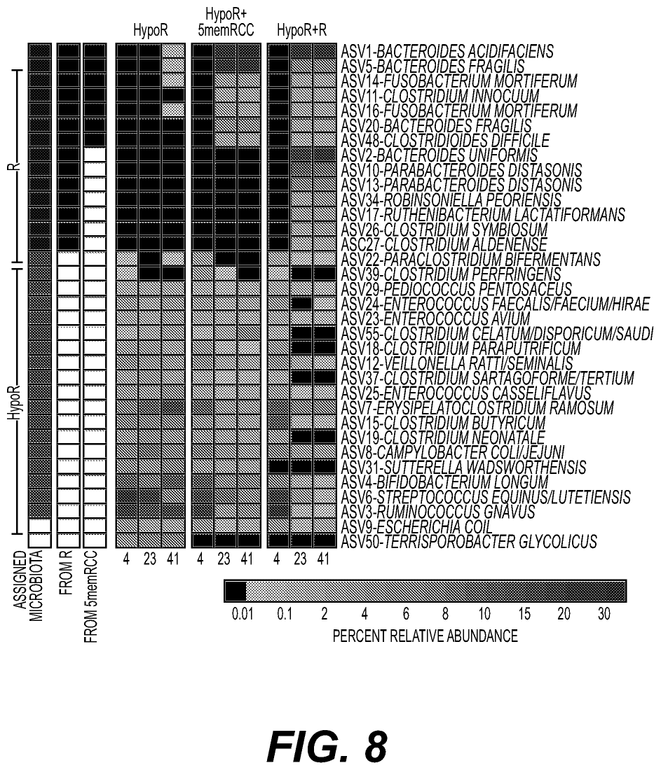

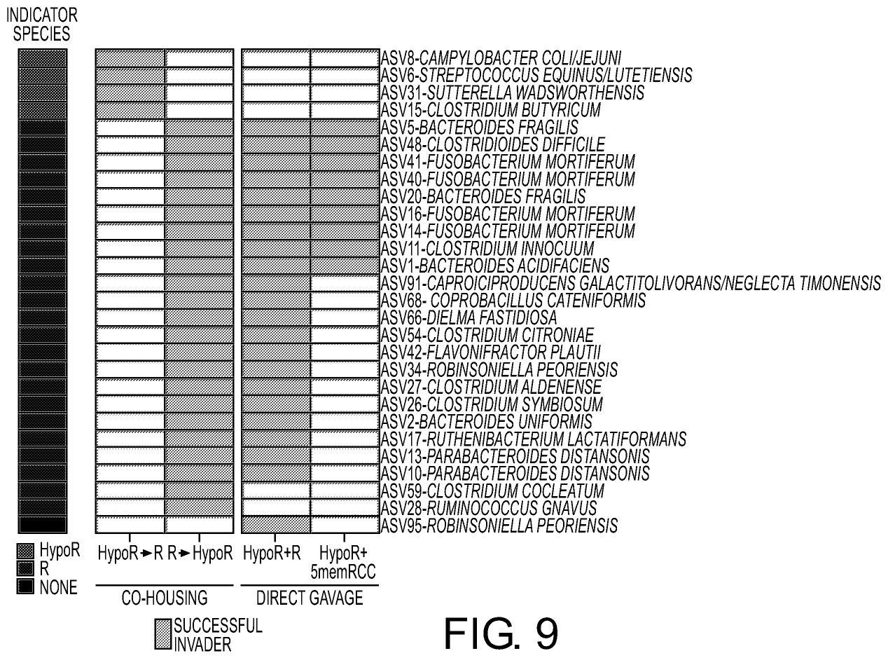

In this follow-on analysis of 16S rDNA datasets from the intact R and 5memRCC probiotic arms, an ASV (amplicon sequence variance) was defined as a successful invader of HypoR-colonized mice if it satisfied two criteria: (i) the ASV increased from a relative abundance <0.1% before gavage to a relative abundance ≥0.1% after gavage in sample from ≥75% of mice in either the HypoR+R or HypoR+5memRCC treatment groups, and (ii) the ASV was not detected at a relative abundance ≥0.1% in more than one HypoR mouse after receiving secondary gavages with the HypoR community. Based on these criteria, 22 ASVs (14 at >1% relative abundance) were identified as invading HypoR-colonized mice after gavage with the R community; all of the ASVs represented in the culture collection were invaders (FIG. 8), and all but one of the 22 (Robinsoniella peoriensis) had been identified previously as successful invaders in the co-housing experiment (FIG. 4C and FIG. 9).

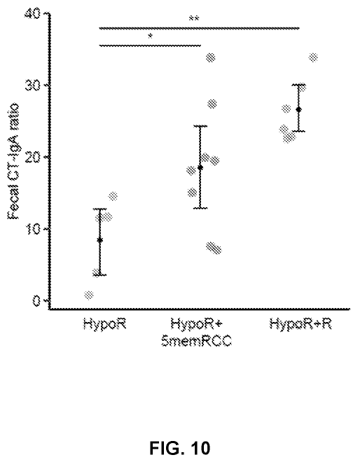

As previously observed, mice in the HypoR+R and HypoR+5memRCC groups exhibited significantly greater mean fecal CT-IgA ratios than HypoR controls [linear models followed by contrasts of marginal means, P=0.001, and P=0.025 respectively (FIG. 10); note that total IgA levels in feces did not differ between groups, either before or after vaccination]. These results demonstrate that five of the bacterial invaders from the R community when administered as a cultured ‘probiotic’ consortium were sufficient to increase the CT-IgA ratio. The CT-IgA ratios in the HypoR+5memRCC mice were negatively correlated with the relative abundances of three HypoR-associated bacterial ASVs; Streptococcus equinusllutetiensis (ASV6, rho=−0.786, P=0.028), Bifidobacterium longum (ASV4, rho=0.762, P=0.037), and a member of the genus Veillonella (ASV12, rho=−0.738, P=0.045).

### Example 4: Microbiota-Associated Metabolites

R Microbiota-Associated Metabolites Correlate with Enhanced CT Specific-IgA Responses

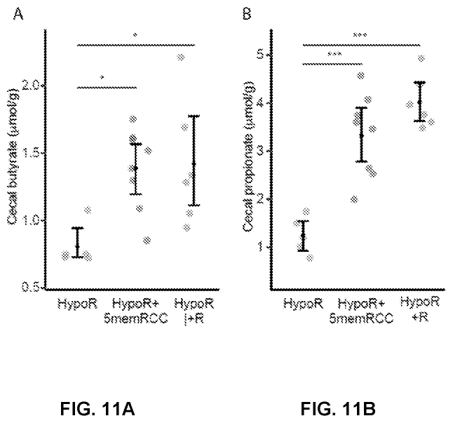

Levels of propionate and butyrate (but not acetate) were significantly higher in fecal samples collected at euthanasia in both the HypoR+R and HypoR+5memRCC treatment groups compared to HypoR controls, whereas succinate was higher only in HypoR+R mice (FIG. 11A, FIG. 11B). These data suggest the 5-member consortium is able to confer similar but not identical changes in fermentative activity on the HypoR community as the full set of invaders from the intact R community. Propionate is produced by four alternative routes in bacteria; the succinate, acrylate, propanediol and dicarboxylic acid pathways. The succinate pathway for propionate fermentation is present in both Bacteroides members of the 5memRCC, while the acrylate pathway that uses lactate is evident in C. difficile. The butyrate fermentation pathway is present in C. innocuum, C. difficile and F. mortiferum (all have a common route for conversion of acetyl-CoA to butyryl-CoA, but distinct reactions for the last step of butyrate production).

To determine whether there are identifiable features of the cecal metabolome associated with the vaccine responsiveness conferred by the intact R community or the 5memRCC, we performed untargeted metabolomics on methanol extracts of cecal contents obtained at sacrifice from each of the mice in the study described in FIG. 5. The analysis was performed using an Agilent 1290 LC system coupled to an Agilent 6545 Q-TOF mass spectrometer. Five μL of each sample for positive ESI ionization were injected onto a BEH C18 column (2.1×150 mm, 1.7 μm, Waters Corp., Milford, Mass.), which was heated to 35° C. The mobile phase consisted of 0.1% formic in water (A) and 0.1% FA in acetonitrile (B), with a flow rate of 0.3 mL/min and a gradient of 5-100% mobile phase B from 0-14 min and then 3 min at 100% B. To provide accurate mass measurements, reference masses m/z 121.0509 and 922.0098 were automatically delivered using dual ESI source during analyses. The mass accuracy of our LC-MS system was ≤4 ppm.

The raw data sets were deconvoluted using MassHunter Profinder B.08.00 software (Agilent Technologies, Santa Clara, Calif.) which generates a list of molecular features. Correlations between intensities of these m/z features and the ratios of fecal anti-CT IgA to total IgA for each mouse were performed; the results revealed two features with m/z of 144.1022 and 235.1078 that had Pearson's r=0.74 (p=0.00003) and 0.72 (p=0.00007) respectively (FIG. 12A and FIG. 12B). m/z 144.1022 was putatively identified as proline betaine based on a monoisotopic mass search in available databases such as METLIN (www.metlin.scripps.edu), KEGG (www.genome.jp/kegg) and HMDB (www.hmdb.ca). Targeted LC/MS/MS revealed that feature m/z 144.1022 and proline betaine had the same retention time and collision induced dissociation (CID) fragmentation pattern, confirming the identity of m/z 144.1022 as proline betaine (FIG. 12A).

The feature with m/z of 235.1078 (FIG. 12B) was putatively identified as a tryptophan-derivative (5-methoxy-DL-tryptophan) based on a monoisotopic mass search. However, its retention time and fragmentation pattern (FIG. 12C) have not been unambiguously assigned at this time. Further studies are required to provide a definitive identification of this metabolite.

Finally, comparison of R community-colonized versus germ-free mice confirmed that these two structures are not intrinsic components of the Nutraceutical Composition I-supplemented M18 diet; rather, their production was dependent on the presence of the microbiota, i.e. they are undetectable in the ceca of germ-free mice (data not shown).

To further explore differences in the metabolic landscape of CT vaccine-responsive mice, and to identify metabolites correlated with the CT-IgA ratio in the context of M18 supplementation, we performed Liquid Chromatography-Quadrupole Time-of-Flight Mass Spectrometry (LC-QTOF-MS) on the cecal contents of mice in a follow-on study. Principal components analysis revealed that the first component of variation separated the R microbiota from the HypoR+R and the HypoR+5memRCC groups and explained 32.0% of the overall variance (FIG. 13A). This first axis was positively correlated with the CT-IgA ratio (Pearson's r=0.67, P=0.002). The second component of variation (explaining 19%) separated the HypoR+5memRCC and HypoR+R communities and, most strongly, the HypoR microbiota. Of the 5,160 analytes (m/z) quantified, 346 had significant positive correlations with CT-IgA ratio (FDR-adjusted P<0.05). Among those with the strongest correlations (r>0.66; n=95), levels of 53 were significantly higher in the cecal contents of both HypoR+R and HypoR+5memRCC animals than in those of HypoR controls, and undetectable in germ-free controls (P<0.05, FDR-corrected Kruskal-Wallis tests followed by pairwise Wilcoxon rank-sums tests corrected by Holm's method; FIG. 13B).

LC-QTOF-MS analysis of the cecal contents of mice in Study 3 (co-housing study) described in FIG. 4A identified 39 supplement-responsive analytes with m/z and retention times matching compounds among the 346 that were correlated with CT-IgA ratios in the Study 4 probiotic gavage experiment; 17 of these were significantly higher in R^{Ch-R}, R^{Ch-HypoR} or HypoR^{Ch-R} mice fed the nutraceutical-supplemented M18 diet, and not detected in their germ-free counterparts (FIG. 13C). These analytes represent biomarkers of supplement-mediated vaccine response in this preclinical model; however, only one (tryptophyl-histidine) could be positively identified by MS. Efforts to determine the contributions of individual members of the 5memRCC consortium to the production of these metabolites based on in vitro culture experiments proved inconclusive (see FIG. 13D). The latter finding highlights the need for additional studies in gnotobiotic mice that compare the efficacy and metabolic output of the full five-member R-derived consortium with that of systemically manipulated versions where smaller subsets, comprised of different combinations of members, are added to the HypoR community.

### Example 5: Encapsulation

Products

The following products (and sources) were used in the coatin/encapsulation processes: Spirulina Powder (Molekula); Compritol 888 ATO (glyceryl dibehenate with a melting range of 65-77° and HLB 2), (Gattefossé); Gelucire 43/01 Pellets (di- and triglyceride esters of fatty acids (C8 to C18), the triester fraction being predominant with a melting temperature of 42-46° and HLB 1) (Gattefossé); Beta-Glucan (Glucan from baker's yeast (S. cerevisiae), USP), (Sigma-Aldrich); Ethanol eurodenatured 99% Technisolv, (VWR chemicals); Ultrapure water

Formulations

Formulation 1: 50% lipid Plurol® Oleique in 700 mL ethanol 70%—2.5 g Spirulina in 335 mL water. The two phases were dissolved separately. Plurol® Oleique phase was first put in a ultrasound bath for 20 minutes and heated for 30 minutes under agitation at 70° C. Afterwards, this phase was dissolved overnight (1000 rpm agitation with a magnetic stirrer). The two phases were then mixed together and passed through the spray dryer.

Formulation 2: 50% Compritol 888 ATO—2.5 g Compritol 888 ATO in 700 mL ethanol 70%-2.5 g Spirulina in 335 mL water. The two phases were dissolved separately. Compritol phase was first put in a ultrasound bath for 20 minutes and heated for 30 minutes under agitation at 70° C. Afterwards, this phase was dissolved overnight (1000 rpm agitation with a magnetic stirrer). The two phases were then mixed together and passed through the spray dryer.

Formulation 3: 50% Gelucire 43/01-2.5 g Gelucire 43/01 Pellets in 700 mL ethanol—2.5 g Spirulina in 335 mL water. The two phases were dissolved separately. Compritol phase was first put in a ultrasound bath for 20 minutes and heated for 30 minutes under agitation at 70° C. The two phases were then mixed together and passed through the spray dryer.

Formulation 4 and 4bis: 25% beta glucan—0.5 g beta glucan in 15 mL NaOH 2M and when completely dissolved, transfer in 700 mL ethanol)—1.5 g Spirulina in 335 mL water

Beta glucan is soluble in NaOH 2M. The two phases were dissolved separately and then mixed together and spray dried.

3 Spray Dryer

A Spray-Dryer SD-OR (Labplant) was used for this study under the following conditions (1,2): —Compressed air pressure: 0.5 bar—Debit: 10 mL/min—T° inlet: 100° C.—T° outlet: around 62° C.

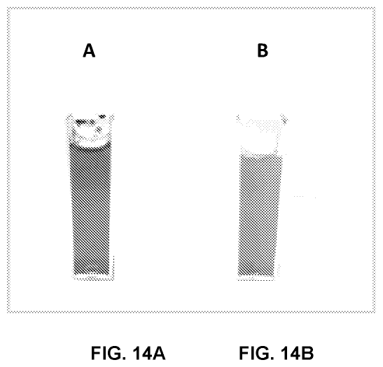

FIG. 14 shows the effect of lipid-coated encapsulation on the color intensity of spirulina. FIG. 14A shows uncoated spirulina, and FIG. 14B shows spirulina coated with 50% lipid encapsulation with Formulation 1 (Plurol® Oleique). As can be seen comparing FIG. 14A to FIG. 14B, the intensity of the spirulina color is significantly masked in the lipid-encapsulated spirulina. Also observed was a significant taste/odor masking effect with the encapsulated spirulina.

Odor and taste masking was also observed with lipid coatings for spray-dried spirulina comprising Formulations 2 and 3.

### Molecular Mimicry Concept

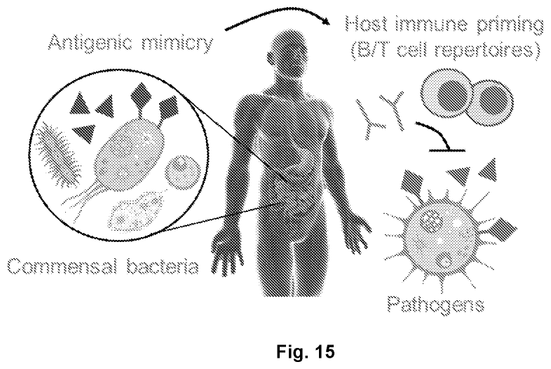

One way to consider the effect of probiotic on a person's immune response is to realize that gut microbiota may play the role of a natural adjuvanted multivalent vaccine. As shown in FIG. 15, microbiota-derived crossreactive antigens may act to prime T cells and/or may act to prime B cells. The primed T and B cells become part of the human T/B cell arsenal and go on to respond to exogenous antigens expressed by pathogens that share sequence/structure homologies with gut microbiota-derived antigens (beneficial antigenic mimicry concept). This may lead to an increased immune response to vaccines.

1. A nutraceutical composition comprising a prebiotic, spirulina and micronutrients, further comprising a probiotic, wherein the probiotic comprises a Bacteroides species, a Fusobacterium species, a Clostridioides species, a Clostridium species, or a combination thereof.

2-12. (canceled)

13. The nutraceutical composition according to claim 1, wherein the probiotic comprises Fusobacterium mortiferum strain 9G6, Bacteroides acidifaciens strain 9G3, Bacteroides fragilis strain 8E3, Clostridium innocuum strain 9H7, Clostridioides difficile strain 9C4 or a combination thereof.

14-18. (canceled)

19. The nutraceutical composition according to claim 1, wherein the prebiotic comprises a cereal selected from the group consisting of: flaxseed, amaranth, rice, oats, teff, bran, barley, wheat, rye, maize, millet, buckwheat, spelt, chia, quinoa or any other grain, or a combination thereof.

20. The nutraceutical composition according to claim 19, wherein the cereal comprises flaxseed and amaranth.

21. The nutraceutical composition according to claim 1, wherein the micronutrients comprise a vitamin and mineral.

22. The nutraceutical composition according to claim 21, wherein the micronutrients comprise i) a vitamin selected from vitamin A (□-carotene, □-carotene, retinol), vitamin B1 (thiamin), vitamin B2 (riboflavin), vitamin B3 (niacin), vitamin B5 (pantothenic acid), vitamin B6 (pyroxidine), vitamin B7 (biotin), vitamin B9 (folate or folic acid), vitamin B12 (cobalamin or cyanocobalamin), vitamin C (ascorbic acid or ascorbate), vitamin D1 (a mixture of lumisterol and califerol), vitamin D2 (ergocalciferol), vitamin D3 (cholecalciferol), vitamin E (□-tocopherol) and vitamin K (phytonadione), and ii) a mineral selected from calcium, chloride, chromium, copper, iodine, iron, magnesium, manganese, molybdenum, phosphorus, potassium, selenium, sodium and zinc.

23. The nutraceutical composition according to claim 22, wherein the micronutrients comprise vitamin B3, vitamin B6, vitamin C, vitamin D3, vitamin E and vitamin B9.

24. The nutraceutical composition according to claim 23, wherein the micronutrients comprise magnesium, selenium and zinc.

25. The nutraceutical composition according to claim 1, wherein the spirulina is encapsulated.

26. The nutraceutical composition according to claim 25, where the encapsulated spirulina is encapsulated with a lipid carrier emulsion.

27. The nutraceutical composition according to claim 26, wherein the lipid carrier emulsion comprises a nonionic emulsifier.

28. The nutraceutical composition according to claim 27, wherein the nonionic emulsifier comprises oleic acid.

29. The nutraceutical composition according to claim 26, wherein the lipid carrier emulsion comprises a microemulsion.

30. The nutraceutical composition according to claim 26, wherein the lipid carrier microemulsion comprises a di- or tri-glyceride.

31. The nutraceutical composition according to claim 29, wherein the lipid carrier microemulsion comprises Plurol® Oleique CC 497 CG, Compritol 888 ATO (glycerol dibehenate), Gelucire 43/01 (di- and tri-glyceride esters of fatty acids).

32. The nutraceutical composition according to claim 26, wherein the lipid carrier emulsion comprises a beta-glucan.

33. The nutraceutical composition according to claim 1, wherein the probiotic is encapsulated.

34. The nutraceutical composition according to claim 33, wherein the encapsulated probiotic is microencapsulated.

35. The nutraceutical composition according to claim 1, wherein the spirulina is present in an amount of 5-15% (dry weight), the flaxseed is present in an amount of 1-5% (dry weight), the amaranth is present in an amount of 5-15% (dry weight) and the micronutrients are present in an amount of 0.02-0.05% (dry weight).

36-40. (canceled)

41. A method of enhancing an immune response to a vaccine or antigen in a human, the method comprising administering an immune enhancing effective amount of a nutraceutical composition comprising: a probiotic; spirulina; cereal comprising flaxseed and amaranth; and micronutrients comprising i) vitamins B3, B6, C, D3, E and B9 and ii) minerals comprising magnesium, selenium and zinc; such that an enhanced immune response to a vaccine or antigen is observed in the human, as measured by an increase in mucosal IgA titer or as measured by an increase in systemic IgG titer, wherein the probiotic comprises a Bacteroides species, a Fusobacterium species, a Clostridioides species, a Clostridium species.

42-90. (canceled)
